(19) United States
(12) Patent Application Publication
(10) Pub. No.: US 2010/0190166 A1
(43) Pub. Date: Jul. 29, 2010

(54) TISSUE REJECTION BIOMARKERS

(75) Inventor: Philip F. Halloran, Edmonton (CA)

Correspondence Address: FISH & RICHARDSON P.C., PO BOX 1022, MINNEAPOLIS, MN, 55440-1022, US

(73) Assignee: The Governors of the University of Alberta

(21) Appl. No.: 12/596,562

(22) PCT Filed: Apr. 18, 2008
(86) PCT No.: PCT/CA08/00719

§ 371 Date: Apr. 6, 2010

Related U.S. Application Data
(60) Provisional application No. 60/925,300, filed on Apr. 19, 2007.

(51) Int. Cl. C12Q 1/68; C12Q 1/02

(52) U.S. Cl. 435/6; 435/29

(57) ABSTRACT

This document relates to methods and materials involved in assessing tissue rejection (e.g., organ rejection) in mammals. For example, methods and materials involved in detecting tissue rejection (e.g., kidney rejection) are provided, as are methods and materials for distinguishing types of tissue rejection (e.g., antibody-mediated rejection versus T cell-mediated rejection) in mammals (e.g., humans).

### CROSS-REFERENCE TO RELATED APPLICATIONS

This application is a claims benefit of priority from U.S. Provisional Application Ser. No. 60/925,300, filed on Apr. 19, 2007.

### TECHNICAL FIELD

This document is related to methods and materials involved in assessing tissue rejection (e.g., organ rejection) in mammals. For example, this document relates to methods and materials involved in detecting tissue rejection (e.g., kidney rejection) and distinguishing types of rejection (e.g., antibody-mediated rejection versus T cell-mediated rejection) in mammals.

### BACKGROUND

The diagnosis of allograft rejection remains an important issue in kidney transplantation. Rejection can manifest as an acute episode or as subtle loss of function, proteinuria, scarring, and graft loss (Meier-Kriesche et al., Am J Transplant, 4(3):378-383 (2004)). Two mechanisms of rejection are recognized in the Banff histologic classification (Solez et al., Am J Transplant, 7(3):518-526 (2007); Racusen et al., Am J Transplant, 4(10):1562-1566 (2004)): T cell mediated rejection (TCMR), diagnosed by scoring interstitial inflammation (i), tubulitis (t), and vasculitis (v); and antibody-mediated rejection (ABMR), a hallmark of which is C4d deposition in peritubular capillaries (Racusen et al., Am J Transplant, 3(6):708-714 (2003)). Histologically, the diagnosis of acute/active ABMR also requires the presence of one of the following lesions: microthrombi, arterial fibrinoid necrosis, glomerulitis, capillaritis, or acute tubular necrosis. In addition, active episodes of antibody mediated immune responses can be superimposed on chronic antibody mediated allograft pathology, which is hallmarked by arterial intimal fibrosis, interstitial fibrosis/tubular atrophy, duplication of the glomerular basement membrane (i.e., transplant glomerulopathy), and lamination of peritubular capillary (PTC) basement membranes. The first of these two findings are nonspecific and an accurate demonstration of PTC basement membrane lamination also requires electron microscopy, which is not routinely performed at most centers.

### SUMMARY

This document provides methods and materials involved in assessing tissue rejection (e.g., organ rejection) in mammals. For example, this document provides methods and materials involved in the early detection of tissue rejection (e.g., kidney rejection). Early diagnosis of patients rejecting transplanted tissue (e.g., a kidney) can allow those patients to be treated sooner, which can increase graft survival rates.

This document also provides methods and materials involved in distinguishing different types of rejection (e.g., distinguishing antibody-mediated rejection (ABMR) from T cell-mediated rejection (TCMR). The differential diagnosis of ABMR and TCMR is complicated, and the complexity is further compounded by the fact that both conditions often occur concurrently. There is a need for the ability to distinguish between different types of rejection such as acute humoral (antibody) mediated rejection and acute cellular rejection (TCMR), particularly since different types of rejection can have different prognoses and can require different therapies. Having the ability to distinguish different types of rejection can help clinicians to determine appropriate treatments for patients undergoing rejection. For example, a clinician who diagnoses a patient as having transplanted tissue that is undergoing antibody-mediated rejection can treat that patient with high-dose intravenous Ig, plasmapheresis, immunoadsorption, or a combination of low-dose intravenous Ig and plasmapheresis, together with more traditional anti-rejection agents.

This document is based in part on the discovery of nucleic acids that are differentially expressed in kidney tissues undergoing antibody-mediated rejection (ABMR), kidney tissues undergoing T cell-mediated rejection (TCMR), and normal nephrectomy tissues. The levels of these nucleic acids and/or polypeptides encoded by these nucleic acids can be used to determine whether tissue transplanted into a mammal is being rejected or is susceptible to being rejected. In addition, the levels of these nucleic acids and/or polypeptides encoded by these nucleic acids can be used to determine whether tissue transplanted into a mammal is undergoing ABMR or TCMR. The levels of multiple nucleic acids or polypeptides can be detected simultaneously using nucleic acid or polypeptide arrays.

In general, this document features a method for detecting tissue rejection. The method comprises, or consists essentially of, determining whether or not tissue transplanted into a mammal contains cells having a transplant rejection profile, where the presence of the cells indicates that the tissue is being rejected. The mammal can be a human. The tissue can be kidney tissue. The tissue can be a kidney. The method can comprise using kidney cells obtained from a biopsy to assess the presence or absence of the transplant rejection profile. The determining step can comprise analyzing nucleic acids. The determining step can comprise analyzing polypeptides.

In another aspect, this document features a method for distinguishing antibody-mediated rejection and T cell-mediated rejection. The method comprises, or consists essentially of, determining whether or not tissue transplanted into a mammal contains cells having an ABMR expression profile, where the presence of the cells indicates that the tissue is undergoing antibody-mediated rejection. The mammal can be a human. The tissue can be kidney tissue. The tissue can be a kidney. The method can comprise using kidney cells obtained from a biopsy to assess the presence or absence of the ABMR expression profile. The determining step can comprise analyzing nucleic acids. The determining step can comprise analyzing polypeptides.

In another aspect, this document features a method for distinguishing antibody-mediated rejection and T cell-mediated rejection. The method comprises, or consists essentially of, determining whether or not tissue transplanted into a mammal contains cells having a TCMR expression profile, where the presence of the cells indicates that the tissue is undergoing T cell-mediated rejection. The mammal can be a human. The tissue can be kidney tissue. The tissue can be a kidney. The method can comprise using kidney cells obtained from a biopsy to assess the presence or absence of the TCMR expression profile. The determining step can comprise analyzing nucleic acids. The determining step can comprise analyzing polypeptides.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a plot of PCA component 1 (58.29% variance) versus PCA component 2 (6.72% variance) from principal components analysis of expression values in ABMR biopsies, TCMR biopsies, and normal nephrectomy (N-Nx) tissues of 220 transcripts that are differentially expressed between ABMR and TCMR biopsies (Table 4).

FIG. 2 is a heat map generated using expression values in ABMR biopsies, TCMR biopsies, and normal nephrectomy (N-Nx) tissues of 220 transcripts that are differentially expressed between ABMR and TCMR biopsies (Table 4). The dendrogram above the heat map shows clustering of ABMR biopsies, TCMR biopsies, and normal nephrectomy specimens.

FIG. 3 contains plots of four gene sets generated by K-means clustering of 220 genes differentially expressed in ABMR and TCMR biopsies (Table 4).

FIG. 4 is a graph plotting fold increase in expression of the indicated NK cell associated transcripts in human CD4, human CD8, and human NK cells relative to corresponding expression levels in normal human nephrectomy tissue.

FIG. 5 is a hierarchical clustering of 165 renal allograft biopsies with available anti-HLA antibody test at time of biopsy using 25 endothelial genes that are changed C4d+ ABMR vs. C4d− TCMR (Pearson correlation, Average linkage). The cluster with high expression of endothelial-transcripts (box) included 31 C4d− Ab+ biopsies clustered together with all 18 C4d+ ABMR biopsies indicating that both groups had similar degree of disturbance in endothelial genes. This cluster also included 31 biopsies with no antibody or C4d with elevated expression of endothelial genes.

FIGS. 6A and 6B contain graphs of graft pathology and survival in 165 biopsies grouped according to the presence of Ab (A), C4d (C), and increased VWF expression (E). The AE or AEC biopsies exhibited increased transplant glomerulopathy (FIG. 6A) and interstitial fibrosis (FIG. 6B) (*p≦0.05). FIG. 6C is a graph plotting death-censored graft survival analysis. The AE or AEC patients had a higher rate of graft loss than A, E, or no AEC patients (Cox regression plots). The hazard ratio (HR) for graft failure was significantly increased in AE or AEC patients, but not in only A or only E patients.

### DETAILED DESCRIPTION

This document provides methods and materials related to assessing tissue rejection (e.g., organ rejection). For example, this document provides methods and materials that can be used to identify a mammal (e.g., a human) as having transplanted tissue that is being rejected. A mammal can be identified as having transplanted tissue that is being rejected if it is determined that the transplanted tissue in the mammal contains cells having a transplant rejection profile, in which genes disclosed herein are over-expressed or under-expressed compared to typical expression levels in non-rejected tissue. For the purposes of this document, the term “transplant rejection profile” as used herein refers to a nucleic or polypeptide profile in a sample where one third or more of the nucleic acids or polypeptides encoded by the nucleic acids listed in Table 2, Table 5, Table 6, and/or Table 9 are present at an elevated level, and/or where one third or more of the nucleic acids or polypeptides encoded by the nucleic acids listed in Table 7 and/or Table 8 are present at a suppressed level compared to the corresponding expression levels in non-rejected tissue. For example, a transplant rejection profile can be a nucleic acid or polypeptide profile in a sample where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 2, Table 5, Table 6, and/or Table 9 are present at an elevated level, and/or where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 7 and/or Table 8 are present at a suppressed level compared to corresponding expression levels in non-rejected tissue.

In some cases, a mammal can be identified as having transplanted tissue that is being rejected if it is determined that the transplanted tissue in the mammal contains cells having a transplant rejection over-expression profile or a transplant rejection under-expression profile, in which genes disclosed herein are over-expressed or under-expressed, respectively, compared to typical expression levels in non-rejected tissue. For the purposes of this document, the term “transplant rejection over-expression profile” as used herein refers to a nucleic acid or polypeptide profile in a sample where one third or more of the nucleic acids or polypeptides encoded by the nucleic acids listed in Table 2, Table 5, Table 6, and/or Table 9 are present at an elevated level compared to the corresponding expression levels in non-rejected tissue. For example, a transplant rejection over-expression profile can be a nucleic acid or polypeptide profile in a sample where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 2, Table 5, Table 6, and/or Table 9 are present at an elevated level. For the purposes of this document, the term “transplant rejection under-expression profile” as used herein refers to a nucleic acid or polypeptide profile in a sample where one third or more of the nucleic acids or polypeptides encoded by the nucleic acids listed in Table 7 and/or Table 8 are present at a suppressed level compared to the corresponding expression levels in non-rejected tissue. For example, transplant rejection under-expression profile can be a nucleic acid or polypeptide profile in a sample where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 7 and/or Table 8 are present at a suppressed level.

This document also provides methods and materials that can be used to determine whether transplanted tissue in a mammal is undergoing antibody-mediated rejection (ABMR) or T cell-mediated rejection (TCMR). For example, transplanted tissue can be identified as undergoing ABMR if it is determined that the transplanted tissue contains cells having an ABMR expression profile, in which genes provided herein are over-expressed or under-expressed compared to typical expression levels in tissues undergoing TCMR. Transplanted tissue can be identified as undergoing TCMR if it is determined that the transplanted tissue contains cells having a TCMR expression profile, in which genes described herein are over-expressed or under-expressed compared to typical expression levels in tissues undergoing ABMR.

For the purposes of this document, the term “ABMR expression profile” as used herein refers to a nucleic acid or polypeptide profile in a sample where one third or more of the nucleic acids or polypeptides encoded by the nucleic acids listed in Table 2, Table 5, and/or Table 9 are present at an elevated level, and/or where one or more than one of the nucleic acids or polypeptides encoded by the nucleic acids listed in Table 7 is present at a suppressed level compared to the corresponding expression levels in tissues undergoing TCMR. For example, an ABMR expression profile can be a nucleic acid or polypeptide profile in a sample where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed Table 2, Table 5, and/or Table 9 are present at an elevated level, and/or where one or more of the nucleic acids or polypeptides encoded by the nucleic acids listed in Table 7 is present at a suppressed level. The nucleic acids listed in Table 10 represent the individual endothelial genes that can be differentially expressed between ABMR and TCMR, most of which (e.g., about 17 of 25) can be present at an elevated level in ABMR.

For the purposes of this document, the term “TCMR expression profile” as used herein refers to a nucleic acid or polypeptide profile in a sample where one third or more of the nucleic acids or polypeptides encoded by the nucleic acids listed in Table 6 are present at an elevated level, and/or where one third or more of the nucleic acids or polypeptides encoded by the nucleic acids listed in Table 8 are present at a suppressed level compared to the corresponding expression levels in tissues undergoing ABMR. For example, a TCMR expression profile can be a nucleic acid or polypeptide profile in a sample where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 6 are present at an elevated level, and/or where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 8 are present at a suppressed level.

In some cases, transplanted tissue can be identified as undergoing ABMR if it is determined that the transplanted tissue contains cells having an ABMR over-expression profile or an ABMR under-expression profile, in which genes provided herein are over-expressed or under-expressed, respectively, compared to typical expression levels in tissues undergoing TCMR. Transplanted tissue can be identified as undergoing TCMR if it is determined that the transplanted tissue contains cells having a TCMR over-expression profile or a TCMR under-expression profile, in which genes provided herein are over-expressed or under-expressed, respectively, compared to typical expression levels in tissues undergoing ABMR.

For the purposes of this document, the term “ABMR over-expression profile” as used herein refers to a nucleic acid or polypeptide profile in a sample where one third or more of the nucleic acids or polypeptides encoded by the nucleic acids listed in Table 2, Table 5, and/or Table 9 are present at an elevated level compared to the corresponding expression levels in tissues undergoing TCMR. For example, an ABMR over-expression profile can be a nucleic acid or polypeptide profile in a sample where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed Table 2, Table 5, and/or Table 9 are present at an elevated level. The nucleic acids listed in Table 10 represent the individual endothelial genes that can be differentially expressed between ABMR and TCMR, most of which (e.g., 17 of 25) can be present at an elevated level in ABMR. For the purposes of this document, the term “ABMR under-expression profile” as used herein refers to a nucleic acid or polypeptide profile in a sample where one or more of the nucleic acids listed in Table 7 is present at a suppressed level compared to corresponding expression levels in tissues undergoing TCMR.

For the purposes of this document, the term “TCMR over-expression profile” as used herein refers to a nucleic acid or polypeptide profile in a sample where one third or more of the nucleic acids or polypeptides encoded by the nucleic acids listed in Table 6 are present at an elevated level as compared to corresponding expression levels in tissues undergoing ABMR. For example, a TCMR over-expression profile can be a nucleic acid or polypeptide profile in a sample where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed Table 6 are present at an elevated level. For the purposes of this document, the term “TCMR under-expression profile” as used herein refers to a nucleic acid or polypeptide profile in a sample where one third or more of the nucleic acids or polypeptides encoded by the nucleic acids listed in Table 8 are present at a suppressed level compared to corresponding expression levels in tissue undergoing ABMR. For example, a TCMR under-expression profile can be a nucleic acid or polypeptide profile in a sample where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 8 are present at a suppressed level.

It will be appreciated that the mean expression level of one third or more (e.g., 34%, 35%, 36%, 37%, 38%, 39%, 40%, 45%, 50%, 55%, 60%, 65%, 70%, 75%, 80%, 85%, 90%, 95%, or 100%) of the nucleic acids or polypeptides encoded by the nucleic acids comprising any of the over-expression or under-expression profiles described herein can be used to identify mammals as having transplanted tissue that is being rejected, or to distinguish ABMR from TCMR. For example, a mammal can be identified as having transplanted tissue that is being rejected if it is determined that the transplanted tissue in the mammal contains cells having a mean transplant rejection over-expression profile. For purposes of this document, the term “mean transplant rejection over-expression profile” refers to a nucleic acid or polypeptide profile in a sample where the mean expression level of one third or more of the nucleic acids or polypeptides encoded by the nucleic acids listed in Table 2, Table 5, Table 6, Table 9, and/or Table 10 is elevated as compared to the corresponding level in unrejected tissue. In some cases, transplanted tissue in a mammal can be identified as undergoing ABMR if it is determined that the transplanted tissue contains cells having a mean ABMR over-expression profile. For purposes of this document, the term “mean ABMR over-expression profile” refers to a nucleic acid or polypeptide profile in a sample where the mean expression level of one third or more of the nucleic acids or polypeptides encoded by the nucleic acids listed in Table 2, Table 5, and/or Table 9 is elevated compared to the corresponding level in tissue undergoing TCMR. The nucleic acids listed in Table 10 represent the individual endothelial genes that can be differentially expressed between ABMR and TCMR, most of which (e.g., about 17 of 25) can be present at an elevated level in ABMR.

The methods and materials provided herein can be used to assess tissue rejection in any mammal such as a human, monkey, horse, dog, cat, cow, pig, mouse, or rat. In addition, the methods and materials provided herein can be used to detect rejection of any type of transplanted tissue including, without limitation, kidney, heart, liver, pancreas, and lung tissue. For example, the methods and materials provided herein can be used to determine whether or not a human who received a kidney transplant is rejecting that transplanted kidney.

Any appropriate sample can be used to determine whether or not transplanted tissue is being rejected in a mammal. For example, biopsy (e.g., punch biopsy, aspiration biopsy, excision biopsy, needle biopsy, or shave biopsy), tissue section, lymph fluid, and blood samples can be used. In some cases, a tissue biopsy sample can be obtained directly from the transplanted tissue. In some cases, a lymph fluid sample can be obtained from one or more lymph vessels that drain from the transplanted tissue. In some cases, a urine sample can be used.

The term “elevated level” as used herein with respect to the level of a nucleic acid or polypeptide encoded by a nucleic acid disclosed herein is any level that is greater than a reference level for that nucleic acid or polypeptide. The term “suppressed level” as used herein with respect to the level of a nucleic acid or polypeptide encoded by a nucleic acid disclosed herein is any level that is lower than a reference level for that nucleic acid or polypeptide. The term “reference level” as used herein with respect to a nucleic acid or polypeptide encoded by a nucleic acid described herein that is being used to identify a mammal as having transplanted tissue that is being rejected can be the level of that nucleic acid or polypeptide typically expressed by cells in tissues that are free of rejection. For example, a reference level of a nucleic acid or polypeptide can be the mean expression level of that nucleic acid or polypeptide, respectively, in cells isolated from kidney tissue that has not been transplanted into a mammal. The term “reference level” as used herein with respect to a nucleic acid or polypeptide encoded by a nucleic acid described herein that is being used to identify transplanted tissue as undergoing ABMR rather than TCMR can be the level of that nucleic acid or polypeptide typically expressed by cells in tissues that are undergoing TCMR. The term “reference level” as used herein with respect to a nucleic acid or polypeptide encoded by a nucleic acid described herein that is being used to identify transplanted tissue as undergoing TCMR rather than ABMR can be the level of that nucleic acid or polypeptide typically expressed by cells in tissues that are undergoing ABMR.

Any appropriate number of samples can be used to determine a reference level. For example, cells obtained from one or more mammals (e.g., at least 5, 10, 15, 25, 50, 75, 100, or more mammals) can be used to determine a reference level. It will be appreciated that levels from comparable samples are used when determining whether or not a particular level is an elevated level. For example, levels from one type of cells are compared to reference levels from the same type of cells. In addition, levels measured by comparable techniques are used when determining whether or not a particular level is an elevated or a suppressed level.

An elevated or suppressed level of a nucleic acid or polypeptide described herein can be any level provided that the level is greater or lower, respectively, than a corresponding reference level for that nucleic acid or polypeptide. For example, an elevated level of a nucleic acid or polypeptide can be 1.1, 1.2, 1.3, 1.4, 1.5, 1.6, 1.7, 1.8, 1.9, 2.0, 2.2, 2.4, 2.6, 2.8, 3, 3.3, 3.6, 4, 4.5, 5, 5.5, 6, 7, 8, 9, 10, 15, 20, or more times greater than a reference level for that nucleic acid or polypeptide, respectively. A suppressed level of a nucleic acid or polypeptide can be 1.1, 1.2, 1.3, 1.4, 1.5, 1.6, 1.7, 1.8, 1.9, 2.0, 2.2, 2.4, 2.6, 2.8, 3, 3.3, 3.6, 4, 4.5, 5, 5.5, 6, 7, 8, 9, 10, 15, 20, or more times lower than a reference level for that nucleic acid or polypeptide, respectively. In addition, a reference level can be any amount. For example, a reference level can be zero. In this case, any level greater than zero would be an elevated level.

Any appropriate method can be used to determine the level of a nucleic acid or polypeptide disclosed herein in a sample from a mammal. For example, quantitative PCR, in situ hybridization, or microarray technology can be used to measure the level of a nucleic acid. In some cases, polypeptide detection methods, such as immunochemistry techniques, can be used to measure the level of a polypeptide encoded by a nucleic acid described herein. For example, antibodies specific for a polypeptide encoded by a nucleic acid disclosed herein can be used to determine the level of the polypeptide in a sample.

Once the level of a nucleic acid or polypeptide encoded by a nucleic acid described herein is determined in a sample from a mammal, then the level can be compared to a reference level for that nucleic acid or polypeptide and used to assess tissue rejection in the mammal. For example, a level of one or more than one nucleic acid or polypeptide encoded by a nucleic acid disclosed herein as being over-expressed in transplanted tissue undergoing ABMR as compared to normal nephrectomy tissue or tissue undergoing TCMR (e.g., a level of one or more than one nucleic acid or polypeptide encoded by a nucleic acid listed in Table 2) that is higher in a sample from a mammal than the corresponding one or more than one reference level can indicate that the mammal comprises transplanted tissue that is being rejected, or that the mammal is susceptible to tissue rejection. In some cases, a level of one or more than one nucleic acid or polypeptide encoded by a nucleic acid disclosed herein as being under-expressed in transplanted tissue undergoing ABMR as compared to normal nephrectomy tissue or tissue undergoing TCMR (e.g., a level of one or more than one nucleic acid or polypeptide encoded by a nucleic acid listed in Table 7) that is lower in a sample from a mammal than the corresponding one or more reference level can indicate that the mammal comprises transplanted tissue that is being rejected or that is susceptible to being rejected. In some cases, a level of one or more than one nucleic acid or polypeptide encoded by a nucleic acid disclosed herein as being differentially expressed in transplanted tissue undergoing ABMR or TCMR (e.g., a level of one or more than one nucleic acid or polypeptide encoded by a nucleic acid listed in Table 4) can be used to distinguish transplanted tissue undergoing ABMR from transplanted tissue undergoing TCMR.

In some cases, the mean (e.g., geometric mean) of the expression levels of more than one nucleic acid or polypeptide encoded by a nucleic acid comprising any of the over-expression or under-expression profiles described herein can be used to assess tissue rejection in a mammal. For example, the mean of the expression levels of one third or more (e.g., 35%, 45%, 55%, 65%, 75%, 85%, 95%, or 100%) of the nucleic acids or polypeptides encoded by the nucleic acids disclosed herein as being over-expressed in tissue undergoing ABMR as compared to tissue undergoing TCMR (e.g., nucleic acids or polypeptides encoded by the nucleic acids listed in Table 2 and Table 10) can be used to distinguish ABMR from TCMR.

The methods and materials provided herein can be used at any time following a tissue transplantation to determine whether or not the transplanted tissue will be rejected. For example, a sample obtained from transplanted tissue at any time following the tissue transplantation can be assessed for the presence of cells expressing an elevated level of one or more nucleic acids or polypeptides encoded by nucleic acids provided herein. In some cases, a sample can be obtained from transplanted tissue 1, 2, 3, 4, 5, 6, 7, 8, 9, 10, 11, 12, or more hours after the transplanted tissue was transplanted. In some cases, a sample can be obtained from transplanted tissue one or more days (e.g., 2, 3, 4, 5, 6, 7, 8, 9, 10, 15, 20, 25, 30, 35, 40, 45, 50, 55, 60, or more days) after the transplanted tissue was transplanted. For example, a sample can be obtained from transplanted tissue 2 to 7 days (e.g., 4 to 6 days) after transplantation and assessed for the presence of cells expressing an elevated level of a nucleic acid or polypeptide encoded by a nucleic acid provided herein. Typically, a biopsy can be obtained any time after transplantation if a patient experiences reduced graft function.

Methods and materials provided herein can be used to assess the effectiveness of a treatment for transplant rejection in a mammal. For example, it can be determined whether or not a mammal having transplanted tissue that is being rejected, and having received a treatment for the transplant rejection, has a mean expression level of nucleic acids or polypeptides encoded by nucleic acids disclosed herein as being over-expressed in rejected tissue as compared to unrejected tissue (e.g., nucleic acids or polypeptides encoded by nucleic acids listed in Table 9) that is lower than a corresponding expression level observed prior to treatment. The presence of the lower level can indicate that the treatment is effective. The absence of the lower level can indicate that the treatment is not effective.

This document also provides methods and materials to assist medical or research professionals in determining whether or not a mammal is undergoing tissue rejection. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of one or more than one nucleic acid or polypeptide encoded by a nucleic acid described herein in a sample, and (2) communicating information about each level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document also provides nucleic acid arrays. The arrays provided herein can be two-dimensional arrays, and can contain at least two different nucleic acid molecules (e.g., at least three, at least five, at least ten, at least 20, at least 30, at least 40, at least 50, or at least 60 different nucleic acid molecules). Each nucleic acid molecule can have any length. For example, each nucleic acid molecule can be between 10 and 250 nucleotides (e.g., between 12 and 200, 14 and 175, 15 and 150, 16 and 125, 18 and 100, 20 and 75, or 25 and 50 nucleotides) in length. In some cases, an array can contain one or more cDNA molecules encoding, for example, partial or entire polypeptides. In addition, each nucleic acid molecule can have any sequence. For example, the nucleic acid molecules of the arrays provided herein can contain sequences that are present within nucleic acids listed in Table 2, Table 5, Table 6, Table 7, Table 8, Table 9, and/or Table 10.

In some cases, at least 25% (e.g., at least 30%, at least 40%, at least 50%, at least 60%, at least 75%, at least 80%, at least 90%, at least 95%, or 100%) of the nucleic acid molecules of an array provided herein contain a sequence that is (1) at least 10 nucleotides (e.g., at least 11, 12, 13, 14, 15, 16, 17, 18, 19, 20, 25, or more nucleotides) in length and (2) at least about 95 percent (e.g., at least about 96, 97, 98, 99, or 100) percent identical, over that length, to a sequence present within a nucleic acid disclosed herein. For example, an array can contain 60 nucleic acid molecules located in known positions, where each of the 60 nucleic acid molecules is 100 nucleotides in length while containing a sequence that is (1) 90 nucleotides is length, and (2) 100 percent identical, over that 90 nucleotide length, to a sequence of a nucleic acid provided herein. A nucleic acid molecule of an array provided herein can contain a sequence present within a nucleic acid described herein where that sequence contains one or more (e.g., one, two, three, four, or more) mismatches.

The nucleic acid arrays provided herein can contain nucleic acid molecules attached to any suitable surface (e.g., plastic, nylon, or glass). In addition, any appropriate method can be used to make a nucleic acid array. For example, spotting techniques and in situ synthesis techniques can be used to make nucleic acid arrays. Further, the methods disclosed in U.S. Pat. Nos. 5,744,305 and 5,143,854 can be used to make nucleic acid arrays.

This document also provides arrays for detecting polypeptides. The arrays provided herein can be two-dimensional arrays, and can contain at least two different polypeptides capable of detecting polypeptides, such as antibodies (e.g., at least three, at least five, at least ten, at least 20, at least 30, at least 40, at least 50, or at least 60 different polypeptides capable of detecting polypeptides). The arrays provided herein also can contain multiple copies of each of many different polypeptides. In addition, the arrays for detecting polypeptides provided herein can contain polypeptides attached to any suitable surface (e.g., plastic, nylon, or glass).

A polypeptide capable of detecting a polypeptide can be naturally occurring, recombinant, or synthetic. The polypeptides immobilized on an array also can be antibodies. An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type, (e.g., IgG, IgM, IgD, IgA or IgY), class (e.g., IgG1, IgG4, or IgA2), or subclass. In addition, an antibody can be from any animal including birds and mammals. For example, an antibody can be a mouse, chicken, human, rabbit, sheep, or goat antibody. Such an antibody can be capable of binding specifically to a polypeptide described herein. The polypeptides immobilized on the array can be members of a family such as a receptor family.

Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a nucleic acid encoding the partial antibody sequence. In some cases, an antibody fragment can be enzymatically or chemically produced by fragmentation of an intact antibody. In addition, numerous antibodies are available commercially. An antibody directed against a polypeptide encoded by a nucleic acid disclosed herein can bind the polypeptide at an affinity of at least 10⁴ mol⁻¹ (e.g., at least 10⁵, 10⁶, 10⁷, 10⁸, 10⁹, 10¹⁰, 10¹¹ or 10¹² mol⁻¹).

Any method can be used to make an array for detecting polypeptides. For example, methods disclosed in U.S. Pat. No. 6,630,358 can be used to make arrays for detecting polypeptides. Arrays for detecting polypeptides can also be obtained commercially, such as from Panomics, Redwood City, Calif.

The invention will be further described in the following examples, which do not limit the scope of the invention described in the claims.

### EXAMPLES

### Example 1

### Identifying Transcripts Characteristic of Renal Antibody-Mediated Rejection

Patients and sample collection: Biopsy specimens were obtained from consenting renal transplant patients undergoing a transplant biopsy for a clinical indication (e.g., deterioration in function, proteinuria, or stable impaired function) as standard of care. Normal kidney tissues were obtained from macroscopically and histologically unaffected areas of the cortex of native nephrectomies performed for renal carcinoma. Biopsies were obtained under ultrasound guidance by spring-loaded needles (ASAP Automatic Biopsy, Microvasive, Watertown, Mass.). In addition to the cores obtained for conventional diagnostic assessment, additional 18-gauge biopsy cores were collected for gene expression analyses. Biopsy cores collected for gene expression analyses were placed in RNALater solution immediately after collection, kept at 4° C. for 4-24 hours, and then stored at −20° C.

Histopathology and clinical data: Paraffin sections were prepared according to Banff criteria (Racusen et al., Kidney Int, 55(2):713-723 (1999)). C4d staining was performed on frozen sections using a monoclonal anti-C4d antibody (Quidel, San Diego, Calif.). All samples had adequate cortical tissue for analysis according to Banff criteria with the exception of two biopsies lacking large arteries. Biopsies were read by a renal pathologist and graded by the Banff classification (Racusen et al., Kidney Int, 55(2):713-723 (1999); Racusen et al., Am J Transplant, 3(6):708-714 (2003); Solez et al., Am J Transplant, 7(3):518-526 (2007)). Clinical data from the time of transplantation to the time of data analysis were collected for each patient and entered into a Laboratory Information Management System (LIMS).

Diagnostic classifications: Histopathologic diagnoses included TCMR, borderline changes, ABMR, mixed TCMR and ABMR, and BK nephropathy. Among patients with histologic evidence of rejection, two levels of diagnosis were defined: 1) the histopathologic classification based on Banff criteria, and 2) the clinical diagnosis of a rejection “episode” based on retrospective assessment of clinical course, independent of the transcriptome analysis. A clinical rejection episode in each case was diagnosed by consensus of two nephrologists. A clinical rejection episode was defined as a decrease in estimated GFR of ≧25% from baseline (up to four months preceding the biopsy when visits were infrequent) and/or an increase in estimated GFR of ≧25% within one month of biopsy in response to anti-rejection therapy. Diagnosis of a clinical rejection episode also required the absence of an alternative explanation for the functional change (e.g., obstruction, infarction, post transplant lymphoproliferative disorder, or calcineurin inhibitor toxicity (CNIT)). Biopsies showing BK nephritis (confirmed by in situ hybridization and/or electron microscopy) were designated as BK (n=6), regardless of whether they also had evidence of TCMR (n=1).

Endothelial cell culture: Human umbilical vein endothelial cells (HUVEC) were isolated from several umbilical cords, pooled, and cultured in complete media (M199 with 20% FCS, penicillin, streptomycin and glutamine) supplemented with ECGS (Invitrogen, Burlington, Ontario, Canada). The cells were passaged and expanded onto gelatin (0.1%)-coated 100 mm dishes (BD Falcon, Mississauga, Ontario, Canada). After three passages, HUVEC cultures were left untreated, or were treated with recombinant human IFN-γ (500 U/mL; eBioscience, San Diego Calif.).

NK cell isolation: NK cells were purified from peripheral blood mononuclear cells (PBMCs) of healthy donors using EasySep® negative selection kits (StemCell, Vancouver, B.C., Canada) according to the manufacturer's instructions. The purity of NK cell isolations varied between 90-98% CD56⁺CD3⁻, as assessed by flow cytometry. Human NK cells were selected from three donors with similarly high ratios of CD56^{lo}/CD56^{bright} NK cells, suggestive of a cytolytic NK phenotype.

RNA preparation and microarray processing: Following homogenization in 0.5 mL of Trizol reagent (Invitrogen, Carlsbad, Calif.), total RNA was extracted and purified using the RNeasy Micro Kit (Qiagen, Ontario, Canada). The average yield was 4 μg/core. RNA (1-2 μg) was labeled using a GeneChip® HT One-Cycle Target Labeling and Control Kit. The quality of labeled cRNA was assessed on an Agilent 2100 Bioanalyzer (Agilent, Palo Alto, Calif.; RNA integrity number >7) before hybridization to a HG_U133_Plus_{—}2.0 GeneChip (Affymetrix, Santa Clara, Calif.). GeneChips were scanned using a GeneArray Scanner (Affymetrix) and processed with GeneChip Operating Software Version 1.4.0 (Affymetrix). Detailed protocols are available in the Affymetrix Technical Manual (on the World Wide Web at affymetrix.com).

Analysis of the transcriptome and the clinical data: Microarrays were pooled into one normalization batch and preprocessed using robust multi-chip averaging (RMA), implemented in GeneSpring GX 7.3.1. Gene expression was analyzed as fold increase over normal controls. Inter-quartile range (IQR, range between the third and first quartiles), non-specific filtering was then used to remove probe sets that have low variability across the entire dataset. Unsupervised methods, such as hierarchical clustering and principal component analysis (PCA), were used to discover classes within the dataset. GeneSpring GX software was used to generate heat maps expressing the relative signal intensities of differentially expressed probe sets. Heat maps were computed based on log of ratio mode, Pearson correlation, or distance as a similarity measure, and average linkage clustering.

Standard class comparison methods were used to generate lists of genes with different levels of expression between ABMR and TCMR (Welch t-test, multiple correction: false discovery rate of 0.05). “Corrected p-values” reported herein refer to false discovery rates (fdr). For example, a corrected p-value of 0.01 signifies that 5% of the probe sets identified as significant at the 0.01 level will, on average, be false positives.

Defining Pathogenesis Based Transcript sets (PBTs): Two sets of cytotoxic T lymphocyte associated transcripts (CATs) were developed: 1) mouse CATs (mCATs; n=236) were defined in rejecting mouse kidney allografts that develop Banff lesions (Einecke et al., Am J Transplant, 5(8):1827-1836 (2005)), and 2) human CATs (hCATs; n=382) were defined in cultured human effector T cells. Sets of transcripts representing interferon gamma (Ifng) effects, referred to as Ifng-dependent rejection induced transcripts (GRITs), also were developed. The “true” tGRITs (n=68) and the “occult” oGRITs (n=326) were both defined in rejecting mouse kidney allografts (Famulski et al., Am J Transplant, 6(6):1342-1354 (2006)). A set of transcripts representing macrophage activation, referred to as Ifng inducible macrophage associated transcripts (IMATs; n=56), were defined in RAW 264.7 cells stimulated with Ifng, and in rejecting mouse kidney allografts. In addition, two sets of transcripts representing renal parenchymal injury were developed: 1) renal transcripts (RTs; n=1481), and 2) a more restricted set of solute carriers (Slcs; n=64), both of which were defined in mouse kidney allografts (Einecke et al., Am J Transplant (2007)). PBTs derived from mouse kidney allografts (Einecke et al., Am J Transplant, 5(8):1827-1836 (2005); Famulski et al., Am J Transplant, 6(6):1342-1354 (2006); Einecke et al., Am J Transplant (2007)) were translated into human ortholog transcripts using information available on the World Wide Web at affymetrix.com.

A set of endothelial cell-associated transcripts (ENDATs; n=323 probe sets corresponding to 118 unique genes) reported to be differentially expressed in endothelial cells compared to non-endothelial cells was identified (Ho et al., Physiol Genomics, 13(3):249-262 (2003); Sengoelge et al., Am J Physiol Renal Physiol, 288(6):F1290-F1300 (2005)).

### Results

Patient demographics: One hundred seventy seven consecutive renal transplant biopsies for unexplained acute or chronic renal dysfunction and/or proteinuria were obtained between 6 days and 31 years post-transplant (median 15.5 months) from 137 consenting recipients, with no exclusions or technical failures. Normal cortical tissue from eight nephrectomies for renal cancer served as controls.

Within all renal allograft biopsies for cause, 40 biopsies from 31 consenting recipients showed histologically proven clinical rejection episodes. Of 40, 15 biopsies were histologically diagnosed as ABMR with diffuse peritubular capillary C4d staining and morphologic signs of tissue injury (peritubular capillaritis and/or glomerulitis and/or acute tubular necrosis-like changes and/or intimal arteritis). All patients with ABMR had circulating anti-HLA antibodies at time of biopsy and had clinical rejection episode. Twenty two allograft biopsies were clinically diagnosed as TCMR episode with a histologic diagnosis of TCMR (n=19) or borderline changes (n=3). The biopsies with mixed ABMR and TCMR (n=3) were excluded from ABMR versus TCMR class comparisons.

ABMR creates a high inflammatory burden in the graft similar to TCMR: PBTs associated with cytotoxic T cells (CATs), macrophage activation (IMATs), and gamma interferon effects (GRITs) were identified as described above. Of 177 biopsies for cause, 171 had available C4d staining. Within these 171 biopsies, geometric means of CATs, GRITs, and IMATs were significantly higher in C4d diffuse+ biopsies compared to C4d− and C4d focal+ biopsies (p<0.05). Moreover, both ABMR and TCMR biopsies showed increased expression of CATs, GRITs, and IMATs compared to biopsies without rejection (p<0.05; Table 1). These results indicate that ABMR creates extensive inflammation in the allograft, as measured by the PBTs, which is quantitatively similar to the inflammation produced by TCMR.

TABLE 1
Mean fold change in expression levels of ENDATs, CATs, GRITs,
and MATs in biopsies for cause versus normal

| | | C4d | C4d | | | |
|---|---|---|---|---|---|---|
| | Normal | diffuse+ | focal+ | C4d− | ABMR | TCMR |
| n | 8 | 17 | 6 | 148 | 15^{a} | 22 |
| ENDATs | 1.0 ± 0.0 | 1.15 ± 0.06** | 0.98 ± 0.08 | 1.04 ± 0.08 | 1.15 ± 0.05** | 1.07 ± 0.07 |
| CATs | 1.0 ± 0.0 | 1.6 ± 0.3* | 1.1 ± 0.2 | 1.3 ± 0.4 | 1.6 ± 0.3 | 2.0 ± 0.6* |
| GRITs | 1.0 ± 0.0 | 2.2 ± 0.4** | 1.3 ± 0.4 | 1.6 ± 0.5 | 2.1 ± 0.4 | 2.4 ± 0.6 |
| MATs | 1.0 ± 0.0 | 2.2 ± 0.4** | 1.3 ± 0.4 | 1.6 ± 0.5 | 2.1 ± 0.4 | 2.4 ± 0.7 |

For gene sets, numbers indicate geometric means of expression values ± SD.
^{a}Mixed ABMR and TCMR cases were excluded;
**p < 0.001;
*p < 0.05.

Expression of Endothelial Cell-Associated Transcripts is increased in ABMR: Alloantibody acting on the microcirculation was hypothesized to alter endothelial gene expression. A set of endothelial cell-associated transcripts (ENDATs) reported to be differentially expressed in endothelial cells when compared to non-endothelial cells (n=118 unique genes) was identified. Of these genes, 19 ENDATs were differentially expressed at an increased level in ABMR versus TCMR (p<0.05; FDR 0.05). Sixteen of these 19 ENDATs were also higher in ABMR than in normal (>1.2 fold). These genes included endothelial markers such as VWF, PECAM1, SELE, CD34, and cadherin 5 (Table 2).

TABLE 2
ENDATs that are selectively increased (n = 19) or decreased (n = 1) in ABMR

| Affymetrix | | Gene | ABMR | | | TCMR | | |
|---|---|---|---|---|---|---|---|---|
| probe set | GI number | symbol | Normalized | StdErr Norm | Raw | Normalized | StdErr Norm | Raw |
| 202112_at | 89191867 | VWF | 6.0372095 | 0.7482771 | 738.30096 | 3.2773373 | 0.4391088 | 428.2687 |
| 212097_at | 11008920 | CAV1 | 5.126014 | 0.47529498 | 857.9012 | 2.9615097 | 0.27006704 | 506.48068 |
| 235489_at | 4569427 | RHOJ | 2.7501938 | 0.24713194 | 85.27414 | 1.5937378 | 0.21596411 | 54.97751 |
| 204677_at | 14589894 | CDH5 | 2.5296934 | 0.22872351 | 201.54375 | 1.5988749 | 0.08614513 | 124.75535 |
| 211340_s_at | 529723 | MCAM | 2.4625611 | 0.21709226 | 476.92596 | 1.743278 | 0.10705583 | 333.32703 |
| 206211_at | 4506870 | SELE | 2.1795537 | 0.36721426 | 55.262226 | 1.1546278 | 0.14220133 | 28.274204 |
| 218736_s_at | 142380801 | PALMD | 2.085945 | 0.15444422 | 182.35475 | 1.3592435 | 0.11957385 | 124.66097 |
| 208981_at | 2705814 | PECAM1 | 1.9562583 | 0.11497954 | 638.3498 | 1.5810047 | 0.0837172 | 518.5426 |
| 226677_at | 12239350 | ZNF521 | 1.5118504 | 0.1153772 | 55.56135 | 0.8744038 | 0.08793866 | 33.970028 |
| 206702_at | 88758595 | TEK | 1.4504417 | 0.11659773 | 275.89493 | 1.0258462 | 0.086594656 | 199.68292 |
| 228665_at | 4312021 | CYYR1 | 1.3985105 | 0.07235072 | 238.68425 | 1.0638516 | 0.0626035 | 183.86978 |
| 209543_s_at | 180108 | CD34 | 1.3809811 | 0.064063214 | 198.86888 | 1.0318205 | 0.04470214 | 149.04498 |
| 220027_s_at | 38570104 | RASIP1 | 1.3279889 | 0.07456124 | 114.53431 | 0.947104 | 0.046973944 | 81.8323 |
| 205522_at | 23397671 | HOXD4 | 1.3048093 | 0.077505335 | 495.27017 | 0.9901667 | 0.050935026 | 376.3266 |
| 221841_s_at | 11599258 | KLF4 | 1.2547917 | 0.11217558 | 318.70065 | 0.83244675 | 0.07891279 | 218.61302 |
| 202052_s_at | 13470085 | RAI14 | 1.186968 | 0.053214792 | 347.62927 | 0.8592701 | 0.060098104 | 258.7931 |
| 203934_at | 11321596 | KDR | 0.7883993 | 0.037757665 | 244.78377 | 0.6164536 | 0.03800948 | 196.96378 |
| 206210_s_at | 4557442 | CETP | 0.7449096 | 0.041633006 | 18.267834 | 1.0892419 | 0.122104675 | 29.588905 |
| 201695_s_at | 4557800 | NP | 0.72189415 | 0.04678709 | 999.8966 | 0.523512 | 0.045881115 | 755.33765 |
| 204041_at | 38202206 | MAOB | 0.7126671 | 0.048660424 | 1031.3046 | 0.5324086 | 0.040042706 | 786.70404 |

The geometric mean of ENDAT expression values correlated significantly (p<0.05) with pathologic features of acute and chronic ABMR, including C4d deposition, peritubular capillaritis, glomerulitis (g), glomerular double contours (cg), and peritubular capillary basement membrane multilayering (PTCBMML; Table 3). These results indicate that increased expression of endothelial genes provides a diagnostic feature of ABMR in renal allografts that distinguishes ABMR from TCMR.

TABLE 3
Correlation coefficients between ENDATs and pathologic features

| | C4d | Peritu- | | | | |
|---|---|---|---|---|---|---|
| | depo- | bular | | | | |
| | sition | capillaritis | g | cg | PTCBMML | t |
| ENDATs | 0.376** | 0.252* | 0.248** | 0.261** | 0.266** | 0.139 |

g, glomerulitis;
cg, glomerular double contours;
PTCBMML, peritubular capillary basement membrane multilayering;
t, tubulitis;
**p < 0.001;
*p < 0.05

Unsupervised Microarray analysis: Differences in gene expression between ABMR and TCMR biopsies were analyzed using an unsupervised bioinformatics approach. Hierarchical clustering and PCA using transcripts that passed the IQR filter (n=1987 probe sets) discriminated biopsies with ABMR and TCMR from controls. However, neither a heat map nor a PCA plot showed a clear discrimination between ABMR and TCMR cases using this large set of genes.

A standard class comparison method (t test and multiple test correction: FDR) was used to identify genes that are important in distinguishing ABMR from TCMR. Three hundred and twenty two probe sets corresponding to 220 unique genes (Table 4) were identified as being differentially expressed between tissues undergoing ABMR and TCMR. Despite overall similarities between ABMR and TCMR (e.g., increased CATs, GRITs, and IMATs), therefore, these two forms of rejection differ significantly in their gene expression profiles.

TABLE 4
Non-redundant list of transcripts differentially expressed between ABMR and TCMR

| Affymetrix | | | Normal | | ABMR | | TCMR | |
|---|---|---|---|---|---|---|---|---|
| probe set | GI number | Gene Symbol | Normalized | Raw | Normalized | Raw | Normalized | Raw |
| 208335_s_at | 42822886 | FY | 0.9839868 | 46.29734 | 6.843061 | 298.8714 | 3.587859 | 190.5149 |
| 203471_s_at | 4505878 | PLEK | 0.9333013 | 42.52589 | 3.079714 | 145.0864 | 4.773315 | 247.7803 |
| 214567_s_at | 38569448 | XCL2 | 1.1083261 | 26.31883 | 3.277935 | 83.67264 | 5.471366 | 192.4745 |
| 220424_at | 7657614 | NPHS2 | 1.100856 | 1354.614 | 0.746183 | 940.7261 | 0.372589 | 556.6851 |
| 223836_at | 13442781 | KSP37 | 0.9589928 | 12.5951 | 2.383065 | 37.25868 | 1.318756 | 18.09349 |
| 204726_at | 61676095 | CDH13 | 0.9933969 | 23.38251 | 2.356655 | 59.57809 | 1.217789 | 30.09278 |
| 205242_at | 5453576 | CXCL13 | 1.022004 | 10.55827 | 3.194202 | 86.12495 | 17.30395 | 524.8595 |
| 206545_at | 5453610 | CD28 | 1.0338584 | 9.904902 | 1.173499 | 11.49033 | 1.84019 | 21.54591 |
| 204677_at | 14589894 | CDH5 | 1.0463884 | 81.46066 | 2.529693 | 201.5438 | 1.598875 | 124.7554 |
| 201721_s_at | 5803055 | LAPTM5 | 0.9756579 | 309.3267 | 3.277659 | 962.5382 | 5.156107 | 1502.53 |
| 217583_at | 13339995 | PAH | 0.85836685 | 261.2564 | 0.52421 | 171.343 | 0.251864 | 99.21797 |
| 203936_s_at | 74272286 | MMP9 | 1.0013682 | 34.79302 | 1.395986 | 55.19832 | 3.016556 | 150.6126 |
| 1555759_a_at | 2905631 | CCL5 | 1.1005995 | 134.9636 | 2.79106 | 366.1121 | 5.562688 | 746.8389 |
| 204774_at | 51511743 | EVI2A | 1.0712854 | 48.37727 | 4.668018 | 218.8261 | 7.204003 | 371.2997 |
| 202345_s_at | 4557580 | FABP5 | 0.90332943 | 32.93694 | 2.97013 | 115.8708 | 4.94858 | 197.0799 |
| 210448_s_at | 1552523 | P2RX5 | 1.0063087 | 44.01488 | 1.491558 | 71.34843 | 2.305007 | 120.3557 |
| 202953_at | 87298827 | C1QB | 0.9100585 | 84.15888 | 4.442529 | 507.3544 | 8.736057 | 871.1847 |
| 202450_s_at | 23110958 | CTSK | 1.0056858 | 106.4038 | 1.620516 | 177.9425 | 2.52399 | 330.2581 |
| 226030_at | 10363759 | ACADSB | 0.9469687 | 1112.143 | 0.583161 | 706.57 | 0.379874 | 498.7044 |
| 215223_s_at | 1331076 | SOD2 | 0.96003467 | 255.0151 | 1.1676 | 327.4565 | 2.282401 | 736.4306 |
| 217147_s_at | 6911580 | TRIM | 0.9862983 | 9.319962 | 1.310662 | 12.9595 | 2.252573 | 27.43823 |
| 221872_at | 4834003 | RARRES1 | 1.3152162 | 55.17371 | 5.702494 | 214.0269 | 10.01571 | 338.3371 |
| 205307_s_at | 52851407 | KMO | 0.9960288 | 188.2769 | 0.743331 | 156.1696 | 0.405907 | 88.25554 |
| 220951_s_at | 20357571 | ACF | 0.8949415 | 383.9646 | 0.631448 | 278.6698 | 0.393373 | 186.0245 |
| 215363_x_at | 6400440 | FOLH1 | 0.855045 | 462.5226 | 0.566397 | 325.6528 | 0.335929 | 206.7323 |
| 201438_at | 55743097 | COL6A3 | 0.8941774 | 136.5098 | 3.118975 | 424.7752 | 5.084672 | 688.9541 |
| 202411_at | 55925613 | IFI27 | 1.0176504 | 204.6181 | 3.699702 | 816.668 | 2.361298 | 527.0441 |
| 205831_at | 31542293 | CD2 | 1.1374997 | 63.0171 | 3.135125 | 179.5427 | 6.763307 | 415.6162 |
| 219840_s_at | 6319191 | TCL6 | 1.1816825 | 66.77751 | 0.71714 | 40.14151 | 0.43689 | 21.70967 |
| 227370_at | 5904131 | KIAA1946 | 1.0409272 | 32.47637 | 2.49728 | 85.36619 | 1.422388 | 47.97654 |
| 1555613_a_at | 26453339 | ZAP70 | 1.078184 | 34.13651 | 1.7597 | 56.85967 | 2.697538 | 107.5365 |
| 1565228_s_at | 598812 | ALB | 1.0892096 | 1057.942 | 0.153343 | 211.5122 | 0.052144 | 94.38497 |
| 206134_at | 16753219 | ADAMDEC1 | 1.0735202 | 9.648431 | 3.514895 | 46.83373 | 14.51142 | 186.6931 |
| 225834_at | 6603583 | MGC57827 | 1.0797637 | 12.85904 | 1.604258 | 19.92049 | 2.581644 | 37.57196 |
| 204852_s_at | 18375657 | PTPN7 | 1.0550075 | 47.17902 | 1.298819 | 60.67023 | 2.042509 | 101.2316 |
| 202917_s_at | 21614543 | S100A8 | 0.7803554 | 143.5425 | 1.025401 | 201.8755 | 1.864872 | 371.3032 |
| 203435_s_at | 116256326 | MME | 0.8400644 | 1811.832 | 0.462317 | 1027.705 | 0.273396 | 642.9791 |
| 228298_at | 10810797 | MGC16044 | 1.0186396 | 36.45953 | 1.359021 | 51.14155 | 2.039441 | 83.54948 |
| 229839_at | 5365256 | MGC45780 | 1.040429 | 14.1213 | 1.308546 | 18.28731 | 2.012937 | 37.05916 |
| 204174_at | 15718674 | ALOX5AP | 1.1168835 | 139.9553 | 1.651618 | 209.2115 | 2.562465 | 342.5766 |
| 228532_at | 7454726 | MGC24133 | 1.0247943 | 89.78178 | 3.524134 | 294.959 | 5.516902 | 471.6237 |
| 220507_s_at | 56550123 | UPB1 | 0.8866968 | 366.9551 | 0.592938 | 275.2925 | 0.356752 | 169.4148 |
| 227794_at | 11444368 | MGC15937 | 0.97580606 | 2176.279 | 0.684482 | 1672.315 | 0.33955 | 908.6433 |
| 202800_at | 34222301 | SLC1A3 | 1.0300162 | 57.89911 | 1.799631 | 120.1976 | 3.970635 | 257.4351 |
| 223922_x_at | 11559215 | MS4A6A | 0.93035257 | 280.416 | 1.915922 | 611.5466 | 3.103309 | 970.5093 |
| 219803_at | 41327750 | ANGPTL3 | 0.8677132 | 620.983 | 0.532892 | 401.278 | 0.272132 | 255.17 |
| 202503_s_at | 71773764 | KIAA0101 | 1.0998269 | 38.02828 | 2.669544 | 95.2258 | 4.527245 | 177.8723 |
| 202196_s_at | 66346687 | DKK3 | 0.98605454 | 48.70658 | 2.230969 | 101.0362 | 1.418349 | 66.87457 |
| 241703_at | 5664001 | RPIB9 | 0.88089246 | 97.8649 | 0.621805 | 68.66184 | 0.391715 | 45.38718 |
| 1555745_a_at | 847819 | LYZ | 1.232983 | 81.49768 | 3.490898 | 251.5294 | 13.8618 | 842.3503 |
| 210279_at | 12005919 | GPR18 | 0.9409255 | 15.13484 | 2.37632 | 41.20143 | 3.684796 | 70.03339 |
| 207651_at | 31377771 | H963 | 1.1338745 | 21.17972 | 2.597098 | 52.32503 | 4.290898 | 98.87205 |
| 204971_at | 61743964 | CSTA | 1.0298616 | 26.64971 | 3.533761 | 106.0114 | 6.214873 | 196.7357 |
| 204115_at | 91807126 | GNG11 | 0.99155474 | 347.1437 | 2.644095 | 922.507 | 1.669506 | 588.0951 |
| 223484_at | 12656020 | NMES1 | 1.0383384 | 31.31874 | 1.677326 | 57.13968 | 2.940447 | 126.3555 |
| 213539_at | 98985799 | CD3D | 1.0824183 | 107.9514 | 1.971853 | 206.828 | 4.008645 | 479.8745 |
| 236430_at | 2718070 | MGC23911 | 0.6723424 | 258.8832 | 0.558427 | 148.9051 | 0.236628 | 79.90163 |
| 224451_x_at | 13543929 | ARHGAP9 | 1.0315305 | 76.00589 | 2.572968 | 194.8184 | 4.228443 | 356.5644 |
| 206030_at | 73622272 | ASPA | 0.91991323 | 1038.339 | 0.673733 | 770.0153 | 0.412192 | 496.3843 |
| 204787_at | 6005957 | Z39IG | 0.9371979 | 104.9149 | 2.030257 | 284.807 | 3.324031 | 409.9208 |
| 206632_s_at | 22907024 | APOBEC3B | 1.0263028 | 12.2339 | 1.417383 | 17.51248 | 2.519598 | 35.85412 |
| 230914_at | 3250320 | HNF4A | 0.9354432 | 386.6656 | 0.589355 | 252.7635 | 0.364981 | 164.314 |
| 212999_x_at | 6663216 | HLA-DQB1 | 1.1951841 | 91.70072 | 3.188465 | 243.708 | 2.005285 | 186.4604 |
| 239929_at | 3058315 | FLJ32569 | 0.8165052 | 59.20098 | 0.570405 | 44.61747 | 0.343548 | 23.64511 |
| 203185_at | 7661963 | RASSF2 | 1.093494 | 35.9015 | 3.042135 | 101.5289 | 4.72178 | 173.1768 |
| 227552_at | 3213287 | Sep1 | 1.0786448 | 23.46265 | 1.34583 | 29.93442 | 2.138103 | 53.89888 |
| 235489_at | 4569427 | RHOJ | 1.0185273 | 31.44351 | 2.750194 | 85.27414 | 1.593738 | 54.97751 |
| 215049_x_at | 312143 | CD163 | 1.0269122 | 102.5058 | 4.276674 | 602.1284 | 8.611414 | 998.7495 |
| 206150_at | 117422442 | TNFRSF7 | 1.0547165 | 55.39373 | 1.871767 | 109.0557 | 3.283592 | 216.1384 |
| 205890_s_at | 50355987 | UBD | 0.9562003 | 159.025 | 5.951329 | 1034.52 | 9.879581 | 2025.316 |
| 229383_at | 4997672 | FLJ20668 | 0.9776168 | 31.77933 | 1.792198 | 64.36266 | 2.771738 | 106.1356 |
| 209827_s_at | 27262654 | IL16 | 1.0056822 | 45.97093 | 1.310199 | 59.85516 | 1.985731 | 97.172 |
| 201761_at | 94721353 | MTHFD2 | 1.1850222 | 79.35938 | 2.806566 | 185.9362 | 4.396514 | 306.1916 |
| 1556737_at | 16550598 | FLJ31222 | 0.9307335 | 229.5063 | 0.57415 | 149.0377 | 0.365948 | 98.07027 |
| 207339_s_at | 4505034 | LTB | 0.9991485 | 99.71491 | 1.349998 | 140.054 | 2.104811 | 246.3901 |
| 222868_s_at | 4435684 | IL18BP | 1.0694878 | 77.51276 | 2.380189 | 178.1787 | 3.772586 | 337.5964 |
| 219505_at | 29029549 | CECR1 | 0.91861933 | 212.7314 | 1.867029 | 425.9912 | 3.481697 | 784.1839 |
| 213603_s_at | 8601388 | RAC2 | 1.177838 | 122.8394 | 4.695681 | 455.1569 | 7.100516 | 759.4375 |
| 211303_x_at | 11078563 | PSMAL/GCPIII | 0.8186896 | 337.3102 | 0.462038 | 201.5225 | 0.270442 | 124.7814 |
| 224997_x_at | 46248265 | H19 | 0.92316514 | 28.47746 | 1.42683 | 50.41486 | 2.544221 | 97.34636 |
| 224840_at | 5132011 | FKBP5 | 0.96449316 | 669.175 | 1.130085 | 875.151 | 2.177175 | 1786.801 |
| 205569_at | 38455384 | LAMP3 | 1.1079527 | 22.42568 | 2.190288 | 51.03503 | 5.066987 | 161.8839 |
| 203914_x_at | 52851450 | HPGD | 1.0108308 | 985.5264 | 0.59918 | 580.9052 | 0.379027 | 412.1836 |
| 209773_s_at | 12804874 | RRM2 | 1.1113011 | 34.56178 | 1.882303 | 61.48326 | 3.501571 | 126.137 |
| 211339_s_at | 399657 | ITK | 1.1025994 | 19.00085 | 2.646908 | 50.14117 | 4.845554 | 110.5974 |
| 210072_at | 2196921 | CCL19 | 1.3582118 | 140.3367 | 3.227925 | 309.07 | 7.862529 | 1071.849 |
| 206682_at | 53832015 | CLECSF14 | 1.1712666 | 42.08045 | 2.016079 | 71.79252 | 3.330614 | 135.6789 |
| 241401_at | 13458148 | FBI4 | 1.0567125 | 125.1322 | 0.861913 | 99.80727 | 0.476213 | 60.14835 |
| 212671_s_at | 13291304 | HLA-DQA1 | 1.0249696 | 599.695 | 2.933556 | 1646.106 | 4.623314 | 2740.361 |
| 226218_at | 8905198 | IL7R | 1.1568464 | 45.93581 | 4.549542 | 166.2807 | 8.132344 | 367.6465 |
| 220485_s_at | 94538334 | SIRPB2 | 1.0537096 | 22.11466 | 1.288721 | 28.16932 | 2.269736 | 57.14916 |
| 216452_at | 10047308 | TRPM3 | 0.9493398 | 342.695 | 0.573144 | 209.0934 | 0.32059 | 127.8845 |
| 203416_at | 91106722 | CD53 | 1.0999957 | 181.1968 | 4.317002 | 704.0284 | 6.530158 | 1092.701 |
| 236285_at | 4683176 | LOC113730 | 1.1004214 | 36.2827 | 1.185836 | 45.7785 | 1.965685 | 73.64101 |
| 215925_s_at | 10281735 | CD72 | 0.9948796 | 25.56977 | 1.720246 | 51.56116 | 2.867165 | 91.58765 |
| 204563_at | 115430250 | SELL | 1.1497816 | 51.02326 | 2.843009 | 134.6903 | 4.967668 | 286.8896 |
| 206214_at | 31543409 | PLA2G7 | 0.9932426 | 10.07087 | 1.361788 | 16.00698 | 2.604655 | 36.77019 |
| 202901_x_at | 33988677 | CTSS | 1.055244 | 20.36709 | 5.090215 | 101.0865 | 8.155622 | 167.5639 |
| 1552755_at | 22749172 | C9orf66 | 0.9390819 | 645.2244 | 0.561941 | 411.0453 | 0.298221 | 238.4532 |
| 208998_at | 2052354 | UCP2 | 1.0408602 | 157.6105 | 2.475807 | 405.3069 | 4.475847 | 774.3045 |
| 244289_at | 3837805 | LOC134466 | 0.94481635 | 64.38574 | 1.607368 | 128.7959 | 0.933539 | 67.17085 |
| 213967_at | 4685862 | LOC138046 | 0.9889094 | 300.984 | 0.645233 | 208.6667 | 0.400118 | 133.9979 |
| 214456_x_at | 758680 | SAA2 | 1.084951 | 38.18197 | 1.117173 | 41.06344 | 1.787348 | 81.21369 |
| 1567628_at | 292154 | CD74 | 0.9653616 | 252.7174 | 1.776663 | 511.1505 | 2.879539 | 880.1469 |
| 230925_at | 3432207 | APBB1IP | 1.0852296 | 245.9594 | 1.578243 | 345.3545 | 2.476961 | 541.4158 |
| 221558_s_at | 9858157 | LEF1 | 1.0079973 | 55.9876 | 1.654941 | 90.35948 | 2.839091 | 191.3492 |
| 219836_at | 56790946 | ZBED2 | 0.9626343 | 12.43085 | 1.128939 | 14.9186 | 1.781355 | 29.31853 |
| 211796_s_at | 3002924 | TCRBV13S1- | 1.0524242 | 74.78034 | 3.812902 | 274.8422 | 7.616922 | 642.8501 |
| | | TCRBJ2S1 | | | | | | |
| 206420_at | 93004093 | IGSF6 | 0.97921413 | 12.82563 | 1.717111 | 26.45523 | 2.905645 | 59.32517 |
| 205404_at | 32455237 | HSD11B1 | 1.0264585 | 40.4002 | 1.465385 | 57.69334 | 2.242145 | 101.3269 |
| 210972_x_at | 338765 | TRA@ | 1.2443407 | 104.7827 | 2.464996 | 205.6086 | 4.757261 | 437.3139 |
| 218796_at | 116686113 | C20orf42 | 0.96682906 | 234.7949 | 0.666706 | 172.3095 | 0.433377 | 112.3206 |
| 203761_at | 113930750 | SLA | 0.9643965 | 100.1526 | 2.308345 | 233.3044 | 3.667605 | 389.269 |
| 218736_s_at | 142380801 | PALMD | 0.9395568 | 80.63562 | 2.085945 | 182.3548 | 1.359244 | 124.661 |
| 207387_s_at | 42794761 | GK | 0.8964044 | 422.3916 | 0.59327 | 289.0754 | 0.386209 | 191.5984 |
| 216920_s_at | 540458 | TRG@ | 0.86961067 | 50.56699 | 1.909283 | 118.0424 | 2.964358 | 207.0307 |
| 206227_at | 51944961 | CILP | 1.1034278 | 47.75473 | 1.197469 | 50.65471 | 1.981107 | 102.7187 |
| 205792_at | 18491001 | WISP2 | 1.0512041 | 30.226 | 1.013041 | 29.21666 | 1.591651 | 59.61497 |
| 239196_at | 3446811 | ANKRD22 | 0.96185005 | 51.43192 | 1.304138 | 75.70496 | 2.165039 | 158.0987 |
| 223599_at | 12407390 | TRIM6 | 1.0254507 | 614.8964 | 1.535024 | 850.6952 | 0.585489 | 356.9431 |
| 221008_s_at | 37574041 | AGXT2L1 | 1.0320444 | 111.4727 | 0.635559 | 67.68906 | 0.370203 | 44.63439 |
| 209671_x_at | 338738 | TRA@; TCRA | 1.1850576 | 99.1357 | 2.052683 | 170.0562 | 3.670259 | 331.1336 |
| 207095_at | 4506972 | SLC10A2 | 0.9339273 | 266.426 | 0.574433 | 176.5741 | 0.308146 | 101.905 |
| 228293_at | 5763726 | LOC91614 | 1.1987456 | 260.9677 | 0.821019 | 163.4289 | 0.494251 | 109.3222 |
| 220646_s_at | 7705573 | KLRF1 | 0.9812503 | 15.76956 | 3.041705 | 52.82301 | 1.997847 | 38.0181 |
| 227346_at | 5109476 | ZNFN1A1 | 1.1123936 | 19.73201 | 4.627268 | 87.366 | 7.509385 | 150.0544 |
| 1553746_a_at | 27734788 | FLJ90579 | 0.9093596 | 85.33128 | 0.808436 | 80.10734 | 0.334232 | 30.60171 |
| 213160_at | 1504001 | DOCK2 | 1.1685091 | 36.95173 | 2.926468 | 93.22987 | 4.666012 | 156.0427 |
| 206878_at | 21536469 | DAO | 0.9553176 | 626.5679 | 0.641769 | 481.6281 | 0.377483 | 285.3359 |
| 207076_s_at | 113204625 | ASS | 0.9819452 | 7807.964 | 0.61104 | 5191.742 | 0.383146 | 3492.054 |
| 202158_s_at | 134152679 | CUGBP2 | 0.94094914 | 17.22038 | 1.685545 | 32.04384 | 2.557743 | 53.30787 |
| 1555349_a_at | 17467029 | ITGB2 | 1.0493845 | 102.626 | 2.911726 | 306.2371 | 4.500004 | 491.2362 |
| 214511_x_at | 182460 | FCGR1A | 1.0162228 | 35.10457 | 3.407321 | 151.6599 | 6.121506 | 261.3835 |
| 210001_s_at | 2443364 | SOCS1 | 1.0746974 | 39.27679 | 1.285837 | 47.52604 | 2.082802 | 86.25831 |
| 206295_at | 27502389 | IL18 | 0.99629647 | 60.68984 | 1.415908 | 94.49966 | 2.249743 | 148.2755 |
| 202075_s_at | 33356542 | PLTP | 1.0250779 | 153.536 | 1.815288 | 235.7251 | 2.989321 | 475.2102 |
| 210644_s_at | 6563041 | LAIR1 | 1.0481691 | 51.18906 | 2.326709 | 122.2881 | 3.846778 | 205.5952 |
| 205758_at | 5855513 | CD8A | 0.9266746 | 56.27314 | 2.553915 | 183.9065 | 5.032148 | 367.1627 |
| 1552807_a_at | 21956185 | SIGLEC10 | 0.9655998 | 38.81354 | 1.646804 | 67.41075 | 2.866463 | 139.2423 |
| 204052_s_at | 8400733 | SFRP4 | 0.9975966 | 13.35091 | 1.369226 | 21.83595 | 2.798249 | 92.7033 |
| 213566_at | 142375535 | RNASE6 | 0.89733326 | 92.27096 | 2.623287 | 281.5954 | 3.957108 | 386.8669 |
| 227677_at | 11597927 | JAK3 | 1.1028577 | 36.79979 | 2.275229 | 76.23268 | 4.017201 | 168.5369 |
| 206211_at | 4506870 | SELE | 1.1235561 | 25.61368 | 2.179554 | 55.26223 | 1.154628 | 28.2742 |
| 211902_x_at | 1100165 | TCRA | 0.9972302 | 109.4112 | 1.648569 | 184.7072 | 2.938053 | 355.9574 |
| 209924_at | 2289718 | CCL18 | 1.0011071 | 46.61441 | 0.924751 | 48.72759 | 1.947654 | 109.9929 |
| 205488_at | 6996012 | GZMA | 1.0221663 | 24.92442 | 4.611108 | 110.5983 | 8.493716 | 214.5559 |
| 222895_s_at | 3058207 | BCL11B | 1.0467502 | 19.35136 | 1.839063 | 35.21671 | 2.958068 | 71.60228 |
| 218232_at | 87298824 | C1QA | 0.90726405 | 145.139 | 3.150009 | 574.4077 | 5.635624 | 970.3551 |
| 210951_x_at | 5410356 | RAB27A | 0.9379082 | 68.68401 | 1.39078 | 107.9919 | 2.139036 | 173.6858 |
| 209795_at | 291897 | CD69 | 1.1266375 | 22.16492 | 2.397995 | 50.53771 | 4.015404 | 91.01402 |
| 236341_at | 5054131 | CTLA4 | 0.9778201 | 11.50043 | 1.222489 | 14.82835 | 1.905454 | 26.35312 |
| 223699_at | 13279082 | CNDP1; CN1; | 0.96248543 | 155.6105 | 0.680131 | 110.4251 | 0.430668 | 74.8282 |
| | | CPGL2; | | | | | | |
| | | HsT2308; | | | | | | |
| | | MGC10825 | | | | | | |
| 206172_at | 26787976 | IL13RA2 | 1.0571084 | 71.9148 | 0.577031 | 37.93879 | 0.379173 | 25.28721 |
| 228056_s_at | 5179093 | NAP1L | 1.0343555 | 18.72981 | 1.544509 | 30.23096 | 2.358335 | 48.95731 |
| 217028_at | 3059119 | CXCR4 | 0.8187881 | 140.6369 | 1.395453 | 260.8801 | 2.668604 | 508.6144 |
| 1552280_at | 19923904 | LOC91937 | 0.982754 | 15.17709 | 0.912522 | 14.15488 | 1.640338 | 38.73613 |
| 202847_at | 66346720 | PCK2 | 0.93247294 | 1126.112 | 0.690823 | 917.0765 | 0.394882 | 547.5838 |
| 225987_at | 2577609 | FLJ23153 | 1.079898 | 91.67351 | 1.431419 | 114.1582 | 2.639602 | 236.331 |
| 212097_at | 11008920 | CAV1 | 1.2176048 | 223.944 | 5.126014 | 857.9012 | 2.96151 | 506.4807 |
| 214059_at | 8366494 | IFI44 | 1.0489386 | 25.44636 | 3.893417 | 119.499 | 2.31677 | 64.51105 |
| 206119_at | 4502406 | BHMT | 0.94769424 | 7676.682 | 0.651079 | 5581.056 | 0.3724 | 3648.368 |
| 215666_at | 1597725 | HLA-DRB3 | 1.0884647 | 10.39229 | 1.299793 | 13.33314 | 2.337008 | 44.49555 |
| 223121_s_at | 5850500 | SFRP2 | 1.0038923 | 37.85015 | 1.458273 | 76.34316 | 3.007463 | 256.6438 |
| 204070_at | 8051633 | RARRES3 | 0.9675206 | 380.7541 | 2.605115 | 996.8603 | 3.931055 | 1686.649 |
| 205692_s_at | 38454325 | CD38 | 0.9900399 | 21.09525 | 2.124981 | 57.51614 | 3.700399 | 108.9046 |
| 209396_s_at | 348911 | CHI3L1 | 1.0709585 | 279.9063 | 0.367349 | 97.05211 | 0.57933 | 173.5339 |
| 210915_x_at | 339011 | TCRB | 1.2324857 | 116.6679 | 4.230006 | 417.7328 | 7.931036 | 891.0783 |
| 220005_at | 29171720 | GPR86 | 1.0503461 | 24.92764 | 2.412599 | 60.09443 | 3.693409 | 100.5417 |
| 230422_at | 5880073 | FPRL2 | 0.98746717 | 39.80808 | 1.576954 | 72.439 | 2.443286 | 105.8494 |
| 206991_s_at | 118572593 | CCR5 | 1.0166248 | 78.53503 | 1.549474 | 133.9208 | 2.796417 | 255.3978 |
| 223582_at | 5902965 | MASS1 | 0.859355 | 229.769 | 0.425515 | 116.2255 | 0.275055 | 81.05927 |
| 204776_at | 40549419 | THBS4 | 0.9939321 | 67.79212 | 0.6966 | 48.48456 | 1.340534 | 179.8929 |
| 206366_x_at | 902001 | XCL1 | 1.0089777 | 28.19423 | 2.925823 | 87.25987 | 5.076693 | 190.3411 |
| 204446_s_at | 62912458 | ALOX5 | 1.0630133 | 57.41484 | 4.182135 | 211.5564 | 6.720236 | 353.6204 |
| 236226_at | 6700716 | BTLA | 0.99204785 | 15.3109 | 1.249579 | 20.10405 | 2.022784 | 37.48604 |
| 205456_at | 50726997 | CD3E | 1.0195386 | 43.15676 | 1.462629 | 65.21019 | 2.29105 | 114.3937 |
| 204489_s_at | 48255934 | CD44 | 0.9554302 | 66.54475 | 1.440001 | 99.11348 | 2.302974 | 182.9427 |
| 1558972_s_at | 27694410 | C6orf190 | 1.0119203 | 8.286964 | 1.42739 | 12.5538 | 2.529968 | 27.52871 |
| 204205_at | 13399303 | APOBEC3G | 1.0467182 | 58.32396 | 4.191694 | 236.3285 | 6.324878 | 373.5277 |
| 210439_at | 5360718 | ICOS | 1.1121944 | 14.55195 | 1.227489 | 16.40603 | 2.033648 | 30.93753 |
| 206930_at | 111038142 | GLYAT | 0.8704912 | 1215.53 | 0.544441 | 807.776 | 0.302378 | 509.76 |
| 236652_at | 6709604 | FLJ39654 | 1.131236 | 468.6387 | 0.709191 | 309.4367 | 0.317941 | 144.9163 |
| 205983_at | 142381187 | DPEP1 | 0.9235758 | 1236.15 | 0.540569 | 767.9671 | 0.307294 | 463.1419 |
| 206914_at | 51593097 | CRTAM | 0.99666804 | 7.899305 | 2.49199 | 21.3833 | 3.891458 | 39.50104 |
| 227560_at | 31068337 | SFXN2 | 1.0103384 | 893.3289 | 0.578462 | 530.699 | 0.374478 | 346.8495 |
| 1569225_a_at | 18204330 | SCML4 | 1.0126843 | 11.34554 | 2.14859 | 26.06536 | 3.264468 | 42.63953 |
| 226303_at | 2716706 | PGM5 | 0.97783506 | 98.28166 | 2.016127 | 202.2085 | 1.245181 | 132.7237 |
| 209685_s_at | 47157321 | PRKCB1 | 1.0279741 | 30.41338 | 3.019459 | 88.41752 | 5.171637 | 172.3315 |
| 206637_at | 125625351 | GPR105 | 1.0413799 | 36.88226 | 2.029468 | 72.0135 | 3.473297 | 171.6284 |
| 203413_at | 5453765 | NELL2 | 1.0321997 | 55.21141 | 1.238331 | 67.07991 | 1.972617 | 122.0421 |
| 205291_at | 23238195 | IL2RB | 1.1322451 | 99.26333 | 1.983277 | 179.032 | 3.517474 | 372.0278 |
| 219655_at | 13376041 | C7orf10 | 0.9589627 | 409.8573 | 0.692279 | 294.1424 | 0.436918 | 201.9344 |
| 209606_at | 431327 | PSCDBP | 1.0218439 | 51.75776 | 2.663854 | 141.7739 | 4.093816 | 240.6958 |
| 205270_s_at | 47078282 | LCP2 | 1.0975697 | 75.86789 | 3.068337 | 222.7177 | 4.647461 | 354.4091 |
| 238531_x_at | 5632820 | OR2I1P; | 0.9962359 | 36.91422 | 1.874806 | 674.23594 | 3.125958 | 179.4888 |
| | | OR2I2; | | | | | | |
| | | OR2I3P; | | | | | | |
| | | OR2I4P; | | | | | | |
| | | HS6M1-14 | | | | | | |
| 232231_at | 7669984 | RUNX2 | 1.0884062 | 23.15499 | 2.424043 | 357.28482 | 4.289425 | 116.2906 |
| 209083_at | 1002922 | CORO1A | 1.0118222 | 59.89005 | 3.301702 | 183.5398 | 4.982481 | 307.2602 |
| 227645_at | 12750213 | P101-PI3K | 1.0029016 | 37.6421 | 1.742109 | 66.37044 | 2.628859 | 103.4917 |
| 228094_at | 4727682 | AMICA | 1.0065863 | 78.90057 | 1.701831 | 135.8982 | 2.685885 | 232.4212 |
| 219607_s_at | 20070327 | MS4A4A | 1.0984327 | 66.85275 | 4.642516 | 351.6191 | 7.692553 | 533.0549 |
| 230278_at | 6505915 | TM7SF1 | 0.89933217 | 84.29004 | 0.653157 | 54.40468 | 0.403656 | 35.67263 |
| 205675_at | 4648246 | MTP | 0.8395886 | 91.71337 | 0.577811 | 65.50542 | 0.356699 | 40.59877 |
| 230061_at | 6835251 | LOC116441 | 0.9316585 | 165.9345 | 2.057199 | 368.0356 | 1.145295 | 216.4135 |
| 204891_s_at | 112789545 | LCK | 1.2384402 | 37.07594 | 3.988754 | 115.242 | 7.200741 | 229.4538 |
| 228375_at | 8908992 | IGSF11 | 0.7941024 | 136.7784 | 0.522075 | 89.16959 | 0.337932 | 59.98387 |
| 216834_at | 123994310 | RGS1 | 1.1030401 | 101.0642 | 0.583049 | 51.1191 | 1.320562 | 118.6257 |
| 226117_at | 1784764 | T2BP | 0.9197388 | 46.56461 | 2.53436 | 124.1546 | 3.856139 | 204.6135 |
| 207085_x_at | 27437028 | CSF2RA | 1.0734321 | 17.69102 | 1.510219 | 27.02881 | 2.450031 | 46.27432 |
| 218883_s_at | 38016934 | KLIP1 | 0.99525696 | 26.59378 | 1.723832 | 49.80592 | 2.65692 | 82.24425 |
| 1555756_a_at | 15986709 | CLECSF12 | 1.0021318 | 12.65696 | 2.403667 | 35.52431 | 3.950642 | 69.25279 |
| 202957_at | 37059786 | HCLS1 | 1.1184098 | 92.00204 | 5.283617 | 430.8466 | 8.275285 | 670.0712 |
| 205666_at | 4503754 | FMO1 | 0.9907695 | 3028.701 | 0.660796 | 2146.079 | 0.391491 | 1401.319 |
| 202112_at | 89191867 | VWF | 1.0672438 | 124.9478 | 6.03721 | 738.301 | 3.277337 | 428.2687 |
| 206666_at | 73747815 | GZMK | 0.94072485 | 56.31801 | 2.513432 | 160.2933 | 5.921053 | 400.0401 |
| 204289_at | 13293226 | C14orf45 | 1.0482601 | 78.70741 | 0.624574 | 49.32914 | 0.410675 | 33.71832 |
| 236295_s_at | 2695005 | NOD3 | 1.0179322 | 29.37287 | 2.533523 | 74.71046 | 4.304266 | 147.2618 |
| 204416_x_at | 51944963 | APOC1 | 0.98057055 | 94.70676 | 1.456043 | 167.3022 | 3.315802 | 362.8008 |
| 219386_s_at | 9910341 | SLAMF8 | 1.0637906 | 29.2116 | 3.085889 | 104.4036 | 6.782425 | 246.1404 |
| 222062_at | 5810334 | IL27RA; CRL1; | 1.0497988 | 19.34444 | 1.9171 | 36.33352 | 3.438613 | 74.11157 |
| | | TCCR; WSX1; | | | | | | |
| | | zcytor1 | | | | | | |
| 201645_at | 4504548 | TNC | 0.95016325 | 113.6369 | 3.792311 | 381.74 | 6.028423 | 607.1257 |

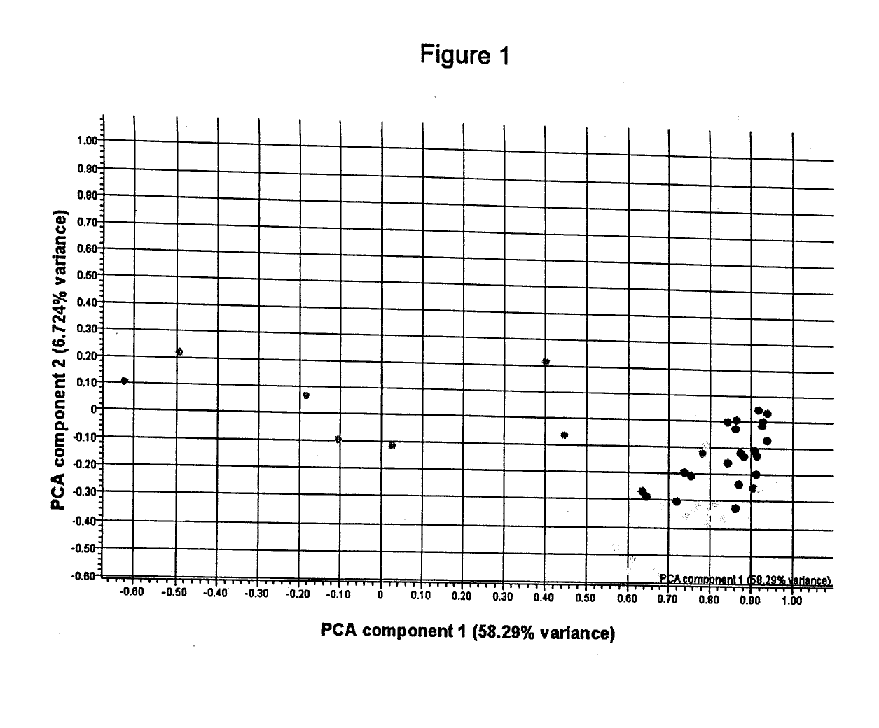

Principal components analysis (PCA) was performed using gene expression values for the 220 genes listed in Table 4 that were differentially expressed between ABMR and TCMR biopsies. The data were mean centered and scaled prior to performing PCA. Results of the analysis indicate that PCA was able to discriminate ABMR biopsies, TCMR biopsies, and normal tissues (FIG. 1).

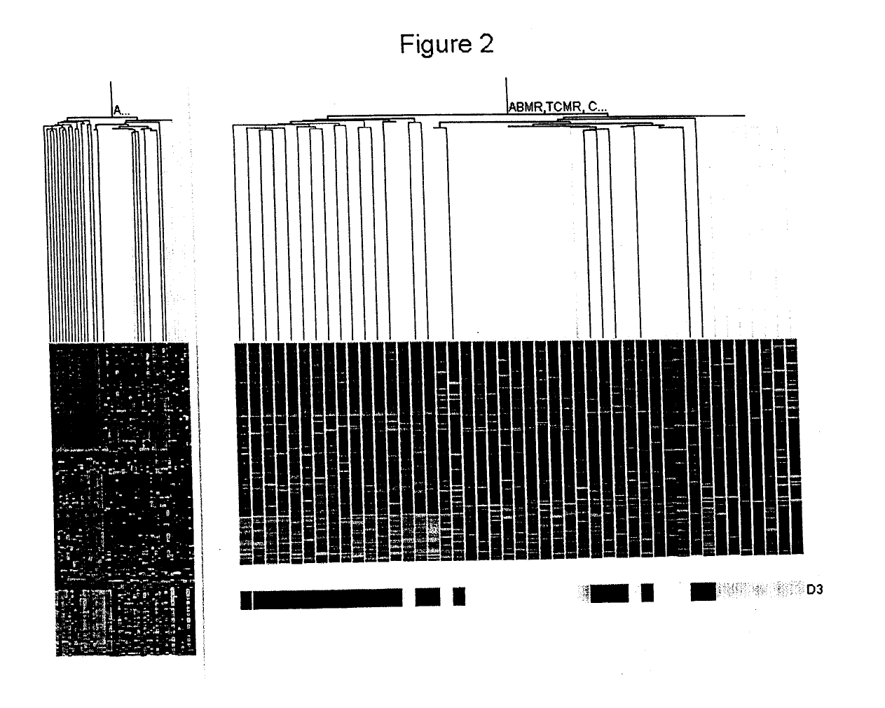

Heirarchical clustering also was performed using gene expression values for the 220 genes listed in Table 4 that were differentially expressed between ABMR and TCMR biopsies. A heat map representing the relative signal intensities of the differentially expressed genes was generated based on distance as a similarity measure and average linkage clustering. Results of these analyses indicate that ABMR cases cluster together and are different from many TCMR cases (FIG. 2).

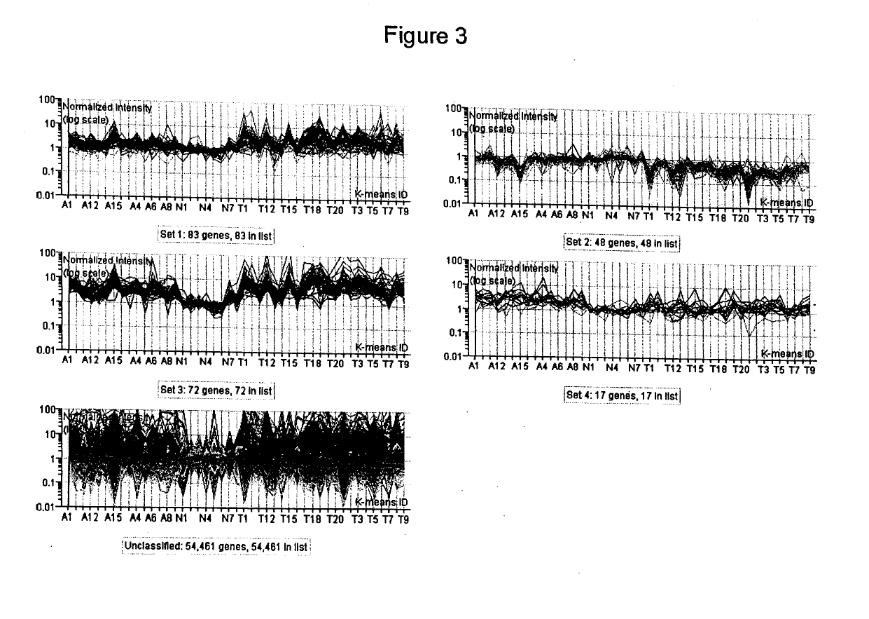

K-means clustering was performed using gene expression values for the 220 genes listed in Table 4 that were differentially expressed between ABMR and TCMR biopsies. K-means clustering generated four gene sets based on distance as a similarity measure. Set 1 (n=83 unique genes) included genes that were selectively increased in TCMR, set 2 (n=48 genes) included genes that were selectively decreased in TCMR, set 3 (n=72 unique genes) mostly included genes with higher expression values in TCMR (n=69 of 72) but also included a few genes with higher expression values in ABMR (n=3 of 72), and set 4 (n=17 unique genes) included genes that were selectively increased in ABMR biopsies (FIG. 3).

Expression levels of the transcripts listed in Table 4 were compared in ABMR biopsies, TCMR biopsies, and normal nephrectomy tissues. Transcripts that showed at least a 1.2 fold increase or decrease in expression relative to the normal nephrectomy tissues were designated as uppers or downers. There were four classes of transcripts within the list of 220 unique genes that differ in expression between ABMR and TCMR: 1) transcripts having increased expression in ABMR (ABMR uppers, n=20 transcripts, Table 5), 2) transcripts having increased expression in TCMR (TCMR uppers, n=151 transcripts, Table 6), 3) transcripts having decreased expression in ABMR (ABMR downers, n=3 genes, Table 7), and 4) transcripts having decreased expression in TCMR (TCMR downers, n=48 genes, Table 8). Examination of individual genes showed that 8 of 20 ABMR uppers are associated with endothelial cells, including DARC, VWF, caveolin 1, cadherin 5, and selectin E (Table 5). Fifty one of 151 TCMR uppers are CATs (Table 6). The top three TCMR upper genes, ADAMDEC1, CXCL13, and lysozyme, are believed to be associated with monocyte/macrophage lineage cells (Table 6). Of 48 genes differentially decreased in TCMR (TCMR downers), 20 are renal transcripts (e.g., SLC10A).

TABLE 5
Transcripts having an expression value that is ≧1.5 fold higher in ABMR than in
TCMR which are also >1.2 fold higher than in Normal

| Affymetrix | | | Normal | | ABMR | | TCMR | |
|---|---|---|---|---|---|---|---|---|
| probe set | GI number | Gene symbol | Normalized | Raw | Normalized | Raw | Normalized | Raw |
| 208335_s_at | 42822886 | FY | 0.9839868 | 46.29734 | 6.8430614 | 298.8714 | 3.587859 | 190.5149 |
| 202112_at | 89191867 | VWF | 1.0672438 | 124.9478 | 6.0372095 | 738.301 | 3.277337 | 428.2687 |
| 212097_at | 11008920 | CAV1 | 1.2176048 | 223.944 | 5.126014 | 857.9012 | 2.96151 | 506.4807 |
| 214059_at | 8366494 | IFI44 | 1.0489386 | 25.44636 | 3.893417 | 119.499 | 2.31677 | 64.51105 |
| 202411_at | 55925613 | IFI27 | 1.0176504 | 204.6181 | 3.6997018 | 816.668 | 2.361298 | 527.0441 |
| 212999_x_at | 6663216 | HLA-DQB1 | 1.1951841 | 91.70072 | 3.1884654 | 243.708 | 2.005285 | 186.4604 |
| 220646_s_at | 7705573 | KLRF1 | 0.9812503 | 15.76956 | 3.0417051 | 52.82301 | 1.997847 | 38.0181 |
| 235489_at | 4569427 | RHOJ | 1.0185273 | 31.44351 | 2.7501938 | 85.27414 | 1.593738 | 54.97751 |
| 204115_at | 91807126 | GNG11 | 0.99155474 | 347.1437 | 2.6440947 | 922.507 | 1.669506 | 588.0951 |
| 204677_at | 14589894 | CDH5 | 1.0463884 | 81.46066 | 2.5296934 | 201.5438 | 1.598875 | 124.7554 |
| 227370_at | 5904131 | KIAA1946 | 1.0409272 | 32.47637 | 2.4972801 | 85.36619 | 1.422388 | 47.97654 |
| 223836_at | 13442781 | KSP37 | 0.9589928 | 12.5951 | 2.3830647 | 37.25868 | 1.318756 | 18.09349 |
| 204726_at | 61676095 | CDH13 | 0.9933969 | 23.38251 | 2.3566554 | 59.57809 | 1.217789 | 30.09278 |
| 202196_s_at | 66346687 | DKK3 | 0.98605454 | 48.70658 | 2.2309694 | 101.0362 | 1.418349 | 66.87457 |
| 206211_at | 4506870 | SELE | 1.1235561 | 25.61368 | 2.1795537 | 55.26223 | 1.154628 | 28.2742 |
| 218736_s_at | 1.42E+08 | PALMD | 0.9395568 | 80.63562 | 2.085945 | 182.3548 | 1.359244 | 124.661 |
| 230061_at | 6835251 | LOC116441 | 0.9316585 | 165.9345 | 2.0571992 | 368.0356 | 1.145295 | 216.4135 |
| 226303_at | 2716706 | PGM5 | 0.97783506 | 98.28166 | 2.0161273 | 202.2085 | 1.245181 | 132.7237 |
| 244289_at | 3837805 | LOC134466 | 0.94481635 | 64.38574 | 1.6073681 | 128.7959 | 0.933539 | 67.17085 |
| 223599_at | 12407390 | TRIM6 | 1.0254507 | 614.8964 | 1.5350237 | 850.6952 | 0.585489 | 356.9431 |

TABLE 6
Transcripts having an expression value that is ≧1.5 fold higher in TCMR than
in ABMR which are also >1.2 fold higher than in Normal

| Affymetrix | | | Normal | | ABMR | | TCMR | |
|---|---|---|---|---|---|---|---|---|
| probe set | GI number | Gene symbol | Normalized | Raw | Normalized | Raw | Normalized | Raw |
| 205242_at | 5453576 | CXCL13 | 1.022004 | 10.55827 | 3.194202 | 86.12495 | 17.303953 | 524.8595 |
| 206134_at | 16753219 | ADAMDEC1 | 1.0735202 | 9.648431 | 3.5148954 | 46.83373 | 14.511418 | 186.6931 |
| 1555745_a_at | 847819 | LYZ | 1.232983 | 81.49768 | 3.4908977 | 251.5294 | 13.861802 | 842.3503 |
| 221872_at | 4834003 | RARRES1 | 1.3152162 | 55.17371 | 5.702494 | 214.0269 | 10.015705 | 338.3371 |
| 205890_s_at | 50355987 | UBD | 0.9562003 | 159.025 | 5.9513288 | 1034.52 | 9.879581 | 2025.316 |
| 202953_at | 87298827 | C1QB | 0.9100585 | 84.15888 | 4.442529 | 507.3544 | 8.736057 | 871.1847 |
| 215049_x_at | 312143 | CD163 | 1.0269122 | 102.5058 | 4.2766743 | 602.1284 | 8.611414 | 998.7495 |
| 205488_at | 6996012 | GZMA | 1.0221663 | 24.92442 | 4.611108 | 110.5983 | 8.493716 | 214.5559 |
| 202957_at | 37059786 | HCLS1 | 1.1184098 | 92.00204 | 5.283617 | 430.8466 | 8.275285 | 670.0712 |
| 202901_x_at | 33988677 | CTSS | 1.055244 | 20.36709 | 5.0902147 | 101.0865 | 8.155622 | 167.5639 |
| 226218_at | 8905198 | IL7R | 1.1568464 | 45.93581 | 4.5495424 | 166.2807 | 8.132344 | 367.6465 |
| 210915_x_at | 339011 | TCRB | 1.2324857 | 116.6679 | 4.2300057 | 417.7328 | 7.9310355 | 891.0783 |
| 210072_at | 2196921 | CCL19 | 1.3582118 | 140.3367 | 3.2279253 | 309.07 | 7.862529 | 1071.849 |
| 219607_s_at | 20070327 | MS4A4A | 1.0984327 | 66.85275 | 4.6425157 | 351.6191 | 7.6925526 | 533.0549 |
| 211796_s_at | 3002924 | TCRBV13S1- | 1.0524242 | 74.78034 | 3.8129015 | 274.8422 | 7.6169224 | 642.8501 |
| | | TCRBJ2S1 | | | | | | |
| 227346_at | 5109476 | ZNFN1A1 | 1.1123936 | 19.73201 | 4.627268 | 87.366 | 7.509385 | 150.0544 |
| 204774_at | 51511743 | EVI2A | 1.0712854 | 48.37727 | 4.668018 | 218.8261 | 7.2040033 | 371.2997 |
| 204891_s_at | 112789545 | LCK | 1.2384402 | 37.07594 | 3.9887538 | 115.242 | 7.200741 | 229.4538 |
| 213603_s_at | 8601388 | RAC2 | 1.177838 | 122.8394 | 4.6956806 | 455.1569 | 7.1005163 | 759.4375 |
| 219386_s_at | 9910341 | SLAMF8 | 1.0637906 | 29.2116 | 3.0858889 | 104.4036 | 6.7824254 | 246.1404 |
| 205831_at | 31542293 | CD2 | 1.1374997 | 63.0171 | 3.1351252 | 179.5427 | 6.7633066 | 415.6162 |
| 204446_s_at | 62912458 | ALOX5 | 1.0630133 | 57.41484 | 4.182135 | 211.5564 | 6.7202363 | 353.6204 |
| 203416_at | 91106722 | CD53 | 1.0999957 | 181.1968 | 4.3170023 | 704.0284 | 6.5301576 | 1092.701 |
| 204205_at | 13399303 | APOBEC3G | 1.0467182 | 58.32396 | 4.191694 | 236.3285 | 6.324878 | 373.5277 |
| 204971_at | 61743964 | CSTA | 1.0298616 | 26.64971 | 3.533761 | 106.0114 | 6.2148733 | 196.7357 |
| 214511_x_at | 182460 | FCGR1A | 1.0162228 | 35.10457 | 3.4073212 | 151.6599 | 6.1215057 | 261.3835 |
| 201645_at | 4504548 | TNC | 0.95016325 | 113.6369 | 3.7923112 | 381.74 | 6.028423 | 607.1257 |
| 206666_at | 73747815 | GZMK | 0.94072485 | 56.31801 | 2.5134323 | 160.2933 | 5.921053 | 400.0401 |
| 218232_at | 87298824 | C1QA | 0.90726405 | 145.139 | 3.1500092 | 574.4077 | 5.6356244 | 970.3551 |
| 1555759_a_at | 2905631 | CCL5 | 1.1005995 | 134.9636 | 2.7910602 | 366.1121 | 5.5626884 | 746.8389 |
| 228532_at | 7454726 | MGC24133 | 1.0247943 | 89.78178 | 3.5241344 | 294.959 | 5.516902 | 471.6237 |
| 214567_s_at | 38569448 | XCL2 | 1.1083261 | 26.31883 | 3.277935 | 83.67264 | 5.471366 | 192.4745 |
| 209685_s_at | 47157321 | PRKCB1 | 1.0279741 | 30.41338 | 3.0194592 | 88.41752 | 5.1716366 | 172.3315 |
| 201721_s_at | 5803055 | LAPTM5 | 0.9756579 | 309.3267 | 3.2776592 | 962.5382 | 5.1561065 | 1502.53 |
| 201438_at | 55743097 | COL6A3 | 0.8941774 | 136.5098 | 3.1189752 | 424.7752 | 5.084672 | 688.9541 |
| 206366_x_at | 902001 | XCL1 | 1.0089777 | 28.19423 | 2.9258227 | 87.25987 | 5.0766926 | 190.3411 |
| 205569_at | 38455384 | LAMP3 | 1.1079527 | 22.42568 | 2.1902876 | 51.03503 | 5.0669866 | 161.8839 |
| 205758_at | 5855513 | CD8A | 0.9266746 | 56.27314 | 2.5539153 | 183.9065 | 5.032148 | 367.1627 |
| 209083_at | 1002922 | CORO1A | 1.0118222 | 59.89005 | 3.3017015 | 183.5398 | 4.9824805 | 307.2602 |
| 204563_at | 115430250 | SELL | 1.1497816 | 51.02326 | 2.8430092 | 134.6903 | 4.9676676 | 286.8896 |
| 202345_s_at | 4557580 | FABP5 | 0.90332943 | 32.93694 | 2.9701295 | 115.8708 | 4.94858 | 197.0799 |
| 211339_s_at | 399657 | ITK | 1.1025994 | 19.00085 | 2.6469078 | 50.14117 | 4.845554 | 110.5974 |
| 203471_s_at | 4505878 | PLEK | 0.9333013 | 42.52589 | 3.0797143 | 145.0864 | 4.7733145 | 247.7803 |
| 210972_x_at | 338765 | TRA@ | 1.2443407 | 104.7827 | 2.4649956 | 205.6086 | 4.7572613 | 437.3139 |
| 203185_at | 7661963 | RASSF2 | 1.093494 | 35.9015 | 3.042135 | 101.5289 | 4.7217803 | 173.1768 |
| 213160_at | 1504001 | DOCK2 | 1.1685091 | 36.95173 | 2.9264684 | 93.22987 | 4.6660123 | 156.0427 |
| 205270_s_at | 47078282 | LCP2 | 1.0975697 | 75.86789 | 3.068337 | 222.7177 | 4.647461 | 354.4091 |
| 212671_s_at | 13291304 | HLA-DQA1 | 1.0249696 | 599.695 | 2.9335558 | 1646.106 | 4.623314 | 2740.361 |
| 202503_s_at | 71773764 | KIAA0101 | 1.0998269 | 38.02828 | 2.6695442 | 95.2258 | 4.5272446 | 177.8723 |
| 1555349_a_at | 17467029 | ITGB2 | 1.0493845 | 102.626 | 2.9117255 | 306.2371 | 4.500004 | 491.2362 |
| 208998_at | 2052354 | UCP2 | 1.0408602 | 157.6105 | 2.4758072 | 405.3069 | 4.4758472 | 774.3045 |
| 201761_at | 94721353 | MTHFD2 | 1.1850222 | 79.35938 | 2.8065658 | 185.9362 | 4.3965135 | 306.1916 |
| 236295_s_at | 2695005 | NOD3 | 1.0179322 | 29.37287 | 2.533523 | 74.71046 | 4.304266 | 147.2618 |
| 207651_at | 31377771 | H963 | 1.1338745 | 21.17972 | 2.5970984 | 52.32503 | 4.2908983 | 98.87205 |
| 232231_at | 7669984 | RUNX2 | 1.0884062 | 23.15499 | 2.4240434 | 57.28482 | 4.289425 | 116.2906 |
| 224451_x_at | 13543929 | ARHGAP9 | 1.0315305 | 76.00589 | 2.572968 | 194.8184 | 4.228443 | 356.5644 |
| 209606_at | 431327 | PSCDBP | 1.0218439 | 51.75776 | 2.663854 | 141.7739 | 4.0938163 | 240.6958 |
| 227677_at | 11597927 | JAK3 | 1.1028577 | 36.79979 | 2.275229 | 76.23268 | 4.017201 | 168.5369 |
| 209795_at | 291897 | CD69 | 1.1266375 | 22.16492 | 2.3979948 | 50.53771 | 4.0154037 | 91.01402 |
| 213539_at | 98985799 | CD3D | 1.0824183 | 107.9514 | 1.9718528 | 206.828 | 4.0086446 | 479.8745 |
| 202800_at | 34222301 | SLC1A3 | 1.0300162 | 57.89911 | 1.7996306 | 120.1976 | 3.970635 | 257.4351 |
| 213566_at | 142375535 | RNASE6 | 0.89733326 | 92.27096 | 2.6232867 | 281.5954 | 3.957108 | 386.8669 |
| 1555756_a_at | 15986709 | CLECSF12 | 1.0021318 | 12.65696 | 2.403667 | 35.52431 | 3.9506416 | 69.25279 |
| 204070_at | 8051633 | RARRES3 | 0.9675206 | 380.7541 | 2.6051154 | 996.8603 | 3.9310553 | 1686.649 |
| 206914_at | 51593097 | CRTAM | 0.99666804 | 7.899305 | 2.4919896 | 21.3833 | 3.8914578 | 39.50104 |
| 226117_at | 1784764 | T2BP | 0.9197388 | 46.56461 | 2.5343602 | 124.1546 | 3.8561392 | 204.6135 |
| 210644_s_at | 6563041 | LAIR1 | 1.0481691 | 51.18906 | 2.3267088 | 122.2881 | 3.846778 | 205.5952 |
| 222868_s_at | 4435684 | IL18BP | 1.0694878 | 77.51276 | 2.3801885 | 178.1787 | 3.7725863 | 337.5964 |
| 205692_s_at | 38454325 | CD38 | 0.9900399 | 21.09525 | 2.1249812 | 57.51614 | 3.7003987 | 108.9046 |
| 220005_at | 29171720 | GPR86 | 1.0503461 | 24.92764 | 2.4125993 | 60.09443 | 3.6934087 | 100.5417 |
| 210279_at | 12005919 | GPR18 | 0.9409255 | 15.13484 | 2.3763196 | 41.20143 | 3.684796 | 70.03339 |
| 209671_x_at | 338738 | TRA@; | 1.1850576 | 99.1357 | 2.0526829 | 170.0562 | 3.6702585 | 331.1336 |
| | | TCRA | | | | | | |
| 203761_at | 113930750 | SLA | 0.9643965 | 100.1526 | 2.3083448 | 233.3044 | 3.6676054 | 389.269 |
| 205291_at | 23238195 | IL2RB | 1.1322451 | 99.26333 | 1.9832765 | 179.032 | 3.517474 | 372.0278 |
| 209773_s_at | 12804874 | RRM2 | 1.1113011 | 34.56178 | 1.882303 | 61.48326 | 3.5015707 | 126.137 |
| 219505_at | 29029549 | CECR1 | 0.91861933 | 212.7314 | 1.8670287 | 425.9912 | 3.4816966 | 784.1839 |
| 206637_at | 125625351 | GPR105 | 1.0413799 | 36.88226 | 2.0294683 | 72.0135 | 3.4732966 | 171.6284 |
| 222062_at | 5810334 | IL27RA; | 1.0497988 | 19.34444 | 1.9170997 | 36.33352 | 3.438613 | 74.11157 |
| | | CRL1; TCCR; | | | | | | |
| | | WSX1; | | | | | | |
| | | zcytor1 | | | | | | |
| 206682_at | 53832015 | CLECSF14 | 1.1712666 | 42.08045 | 2.0160785 | 71.79252 | 3.3306136 | 135.6789 |
| 204787_at | 6005957 | Z39IG | 0.9371979 | 104.9149 | 2.0302565 | 284.807 | 3.3240314 | 409.9208 |
| 204416_x_at | 51944963 | APOC1 | 0.98057055 | 94.70676 | 1.4560428 | 167.3022 | 3.3158023 | 362.8008 |
| 206150_at | 117422442 | TNFRSF7 | 1.0547165 | 55.39373 | 1.8717674 | 109.0557 | 3.2835915 | 216.1384 |
| 1569225_a_at | 18204330 | SCML4 | 1.0126843 | 11.34554 | 2.14859 | 26.06536 | 3.2644682 | 42.63953 |
| 238531_x_at | 5632820 | OR2I1P; | 0.9962359 | 36.91422 | 1.8748063 | 74.23594 | 3.1259577 | 179.4888 |
| | | OR2I2; | | | | | | |
| | | OR2I3P; | | | | | | |
| | | OR2I4P; | | | | | | |
| | | HS6M1-14 | | | | | | |
| 223922_x_at | 11559215 | MS4A6A | 0.93035257 | 280.416 | 1.9159218 | 611.5466 | 3.1033087 | 970.5093 |
| 203936_s_at | 74272286 | MMP9 | 1.0013682 | 34.79302 | 1.395986 | 55.19832 | 3.0165558 | 150.6126 |
| 223121_s_at | 5850500 | SFRP2 | 1.0038923 | 37.85015 | 1.4582728 | 76.34316 | 3.007463 | 256.6438 |
| 202075_s_at | 33356542 | PLTP | 1.0250779 | 153.536 | 1.8152878 | 235.7251 | 2.989321 | 475.2102 |
| 216920_s_at | 540458 | TRG@ | 0.86961067 | 50.56699 | 1.909283 | 118.0424 | 2.964358 | 207.0307 |
| 222895_s_at | 3058207 | BCL11B | 1.0467502 | 19.35136 | 1.8390627 | 35.21671 | 2.958068 | 71.60228 |
| 223484_at | 12656020 | NMES1 | 1.0383384 | 31.31874 | 1.6773262 | 57.13968 | 2.940447 | 126.3555 |
| 211902_x_at | 1100165 | TCRA | 0.9972302 | 109.4112 | 1.6485691 | 184.7072 | 2.9380527 | 355.9574 |
| 206420_at | 93004093 | IGSF6 | 0.97921413 | 12.82563 | 1.7171105 | 26.45523 | 2.9056447 | 59.32517 |
| 1567628_at | 292154 | CD74 | 0.9653616 | 252.7174 | 1.7766625 | 511.1505 | 2.8795385 | 880.1469 |
| 215925_s_at | 10281735 | CD72 | 0.9948796 | 25.56977 | 1.720246 | 51.56116 | 2.8671653 | 91.58765 |
| 1552807_a_at | 21956185 | SIGLEC10 | 0.9655998 | 38.81354 | 1.6468035 | 67.41075 | 2.8664632 | 139.2423 |
| 221558_s_at | 9858157 | LEF1 | 1.0079973 | 55.9876 | 1.6549408 | 90.35948 | 2.839091 | 191.3492 |
| 204052_s_at | 8400733 | SFRP4 | 0.9975966 | 13.35091 | 1.3692255 | 21.83595 | 2.798249 | 92.7033 |
| 206991_s_at | 118572593 | CCR5 | 1.0166248 | 78.53503 | 1.5494735 | 133.9208 | 2.7964168 | 255.3978 |
| 229383_at | 4997672 | FLJ20668 | 0.9776168 | 31.77933 | 1.7921976 | 64.36266 | 2.7717378 | 106.1356 |
| 1555613_a_at | 26453339 | ZAP70 | 1.078184 | 34.13651 | 1.7596996 | 56.85967 | 2.6975384 | 107.5365 |
| 228094_at | 4727682 | AMICA | 1.0065863 | 78.90057 | 1.7018309 | 135.8982 | 2.685885 | 232.4212 |
| 217028_at | 3059119 | CXCR4 | 0.8187881 | 140.6369 | 1.3954531 | 260.8801 | 2.6686037 | 508.6144 |
| 218883_s_at | 38016934 | KLIP1 | 0.99525696 | 26.59378 | 1.723832 | 49.80592 | 2.6569197 | 82.24425 |
| 225987_at | 2577609 | FLJ23153 | 1.079898 | 91.67351 | 1.4314187 | 114.1582 | 2.639602 | 236.331 |
| 227645_at | 12750213 | P101-PI3K | 1.0029016 | 37.6421 | 1.7421094 | 66.37044 | 2.6288586 | 103.4917 |
| 206214_at | 31543409 | PLA2G7 | 0.9932426 | 10.07087 | 1.3617884 | 16.00698 | 2.604655 | 36.77019 |
| 225834_at | 6603583 | MGC57827 | 1.0797637 | 12.85904 | 1.6042576 | 19.92049 | 2.5816443 | 37.57196 |
| 204174_at | 15718674 | ALOX5AP | 1.1168835 | 139.9553 | 1.6516178 | 209.2115 | 2.5624645 | 342.5766 |
| 202158_s_at | 134152679 | CUGBP2 | 0.94094914 | 17.22038 | 1.6855452 | 32.04384 | 2.5577433 | 53.30787 |
| 224997_x_at | 46248265 | H19 | 0.92316514 | 28.47746 | 1.4268304 | 50.41486 | 2.5442207 | 97.34636 |
| 1558972_s_at | 27694410 | C6orf190 | 1.0119203 | 8.286964 | 1.4273896 | 12.5538 | 2.5299683 | 27.52871 |
| 202450_s_at | 23110958 | CTSK | 1.0056858 | 106.4038 | 1.6205155 | 177.9425 | 2.5239904 | 330.2581 |
| 206632_s_at | 22907024 | APOBEC3B | 1.0263028 | 12.2339 | 1.4173828 | 17.51248 | 2.5195975 | 35.85412 |
| 230925_at | 3432207 | APBB1IP | 1.0852296 | 245.9594 | 1.5782428 | 345.3545 | 2.4769614 | 541.4158 |
| 207085_x_at | 27437028 | CSF2RA | 1.0734321 | 17.69102 | 1.5102185 | 27.02881 | 2.4500306 | 46.27432 |
| 230422_at | 5880073 | FPRL2 | 0.98746717 | 39.80808 | 1.5769535 | 72.439 | 2.4432864 | 105.8494 |
| 228056_s_at | 5179093 | NAP1L | 1.0343555 | 18.72981 | 1.5445085 | 30.23096 | 2.3583345 | 48.95731 |
| 215666_at | 1597725 | HLA-DRB3 | 1.0884647 | 10.39229 | 1.2997934 | 13.33314 | 2.337008 | 44.49555 |
| 210448_s_at | 1552523 | P2RX5 | 1.0063087 | 44.01488 | 1.4915577 | 71.34843 | 2.3050065 | 120.3557 |
| 204489_s_at | 48255934 | CD44 | 0.9554302 | 66.54475 | 1.4400012 | 99.11348 | 2.3029742 | 182.9427 |
| 205456_at | 50726997 | CD3E | 1.0195386 | 43.15676 | 1.4626292 | 65.21019 | 2.2910495 | 114.3937 |
| 215223_s_at | 1331076 | SOD2 | 0.96003467 | 255.0151 | 1.1675997 | 327.4565 | 2.282401 | 736.4306 |
| 220485_s_at | 94538334 | SIRPB2 | 1.0537096 | 22.11466 | 1.2887205 | 28.16932 | 2.2697358 | 57.14916 |
| 217147_s_at | 6911580 | TRIM | 0.9862983 | 9.319962 | 1.3106617 | 12.9595 | 2.2525728 | 27.43823 |
| 206295_at | 27502389 | IL18 | 0.99629647 | 60.68984 | 1.4159079 | 94.49966 | 2.2497425 | 148.2755 |
| 205404_at | 32455237 | HSD11B1 | 1.0264585 | 40.4002 | 1.4653854 | 57.69334 | 2.2421446 | 101.3269 |
| 224840_at | 5132011 | FKBP5 | 0.96449316 | 669.175 | 1.1300849 | 875.151 | 2.1771753 | 1786.801 |
| 239196_at | 3446811 | ANKRD22 | 0.96185005 | 51.43192 | 1.3041382 | 75.70496 | 2.165039 | 158.0987 |
| 210951_x_at | 5410356 | RAB27A | 0.9379082 | 68.68401 | 1.3907796 | 107.9919 | 2.139036 | 173.6858 |
| 227552_at | 3213287 | Sep1 | 1.0786448 | 23.46265 | 1.3458303 | 29.93442 | 2.1381025 | 53.89888 |
| 207339_s_at | 4505034 | LTB | 0.9991485 | 99.71491 | 1.3499978 | 140.054 | 2.104811 | 246.3901 |
| 210001_s_at | 2443364 | SOCS1 | 1.0746974 | 39.27679 | 1.2858367 | 47.52604 | 2.0828023 | 86.25831 |
| 204852_s_at | 18375657 | PTPN7 | 1.0550075 | 47.17902 | 1.298819 | 60.67023 | 2.0425093 | 101.2316 |
| 228298_at | 10810797 | MGC16044 | 1.0186396 | 36.45953 | 1.3590213 | 51.14155 | 2.0394413 | 83.54948 |
| 210439_at | 5360718 | ICOS | 1.1121944 | 14.55195 | 1.2274885 | 16.40603 | 2.0336483 | 30.93753 |
| 236226_at | 6700716 | BTLA | 0.99204785 | 15.3109 | 1.2495794 | 20.10405 | 2.0227842 | 37.48604 |
| 229839_at | 5365256 | MGC45780 | 1.040429 | 14.1213 | 1.3085461 | 18.28731 | 2.0129366 | 37.05916 |
| 209827_s_at | 27262654 | IL16 | 1.0056822 | 45.97093 | 1.3101991 | 59.85516 | 1.9857306 | 97.172 |
| 206227_at | 51944961 | CILP | 1.1034278 | 47.75473 | 1.197469 | 50.65471 | 1.9811074 | 102.7187 |
| 203413_at | 5453765 | NELL2 | 1.0321997 | 55.21141 | 1.2383306 | 67.07991 | 1.9726167 | 122.0421 |
| 236285_at | 4683176 | LOC113730 | 1.1004214 | 36.2827 | 1.1858362 | 45.7785 | 1.965685 | 73.64101 |
| 209924_at | 2289718 | CCL18 | 1.0011071 | 46.61441 | 0.9247505 | 48.72759 | 1.9476541 | 109.9929 |
| 236341_at | 5054131 | CTLA4 | 0.9778201 | 11.50043 | 1.2224894 | 14.82835 | 1.9054543 | 26.35312 |
| 202917_s_at | 21614543 | S100A8 | 0.7803554 | 143.5425 | 1.0254006 | 201.8755 | 1.8648721 | 371.3032 |
| 206545_at | 5453610 | CD28 | 1.0338584 | 9.904902 | 1.1734991 | 11.49033 | 1.8401897 | 21.54591 |
| 214456_x_at | 758680 | SAA2 | 1.084951 | 38.18197 | 1.1171734 | 41.06344 | 1.7873484 | 81.21369 |
| 219836_at | 56790946 | ZBED2 | 0.9626343 | 12.43085 | 1.1289392 | 14.9186 | 1.781355 | 29.31853 |
| 1552280_at | 19923904 | LOC91937 | 0.982754 | 15.17709 | 0.91252226 | 14.15488 | 1.6403375 | 38.73613 |
| 205792_at | 18491001 | WISP2 | 1.0512041 | 30.226 | 1.0130408 | 29.21666 | 1.5916508 | 59.61497 |
| 204776_at | 40549419 | THBS4 | 0.9939321 | 67.79212 | 0.69659984 | 48.48456 | 1.3405335 | 179.8929 |

TABLE 7
Transcripts having an expression value that is <1.5 fold lower in ABMR than
in TCMR which are also <1.2 fold lower than in Normal

| Affymetrix | | | Normal | | ABMR | | TCMR | |
|---|---|---|---|---|---|---|---|---|
| probe set | GI number | Gene symbol | Normalized | Raw | Normalized | Raw | Normalized | Raw |
| 204776_at | 40549419 | THBS4 | 0.9939321 | 67.79212 | 0.69659984 | 48.48456 | 1.3405335 | 179.8929 |
| 216834_at | 123994310 | RGS1 | 1.1030401 | 101.0642 | 0.5830492 | 51.1191 | 1.3205618 | 118.6257 |
| 209396_s_at | 348911 | CHI3L1 | 1.0709585 | 279.9063 | 0.36734888 | 97.05211 | 0.5793302 | 173.5339 |

TABLE 8
Transcripts having an expression value that is <1.5 fold lower in TCMR than
in ABMR which are also <1.2 fold lower than in Normal

| Affymetrix | | | Normal | | ABMR | | TCMR | |
|---|---|---|---|---|---|---|---|---|
| probe set | GI number | Gene symbol | Normalized | Raw | Normalized | Raw | Normalized | Raw |
| 1565228_s_at | 598812 | ALB | 1.0892096 | 1057.942 | 0.1533426 | 211.5122 | 0.0521442 | 94.38497 |
| 236430_at | 2718070 | MGC23911 | 0.6723424 | 258.8832 | 0.5584273 | 148.9051 | 0.2366277 | 79.90163 |
| 217583_at | 13339995 | PAH | 0.85836685 | 261.2564 | 0.5242102 | 171.343 | 0.2518643 | 99.21797 |
| 211303_x_at | 11078563 | PSMAL/GCPIII | 0.8186896 | 337.3102 | 0.462038 | 201.5225 | 0.270442 | 124.7814 |
| 219803_at | 41327750 | ANGPTL3 | 0.8677132 | 620.983 | 0.5328921 | 401.278 | 0.2721316 | 255.17 |
| 203435_s_at | 1.16E+08 | MME | 0.8400644 | 1811.832 | 0.462317 | 1027.705 | 0.2733957 | 642.9791 |
| 223582_at | 5902965 | MASS1 | 0.859355 | 229.769 | 0.4255154 | 116.2255 | 0.2750551 | 81.05927 |
| 1552755_at | 22749172 | C9orf66 | 0.9390819 | 645.2244 | 0.5619411 | 411.0453 | 0.298221 | 238.4532 |
| 206930_at | 1.11E+08 | GLYAT | 0.8704912 | 1215.53 | 0.5444414 | 807.776 | 0.3023777 | 509.76 |
| 205983_at | 1.42E+08 | DPEP1 | 0.9235758 | 1236.15 | 0.5405689 | 767.9671 | 0.3072944 | 463.1419 |
| 207095_at | 4506972 | SLC10A2 | 0.9339273 | 266.426 | 0.5744327 | 176.5741 | 0.3081456 | 101.905 |
| 236652_at | 6709604 | FLJ39654 | 1.131236 | 468.6387 | 0.709191 | 309.4367 | 0.3179412 | 144.9163 |
| 216452_at | 10047308 | TRPM3 | 0.9493398 | 342.695 | 0.5731444 | 209.0934 | 0.3205895 | 127.8845 |
| 1553746_a_at | 27734788 | FLJ90579 | 0.9093596 | 85.33128 | 0.8084361 | 80.10734 | 0.334232 | 30.60171 |
| 215363_x_at | 6400440 | FOLH1 | 0.855045 | 462.5226 | 0.566397 | 325.6528 | 0.3359294 | 206.7323 |
| 228375_at | 8908992 | IGSF11 | 0.7941024 | 136.7784 | 0.5220749 | 89.16959 | 0.3379324 | 59.98387 |
| 227794_at | 11444368 | MGC15937 | 0.97580606 | 2176.279 | 0.6844823 | 1672.315 | 0.3395499 | 908.6433 |
| 239929_at | 3058315 | FLJ32569 | 0.8165052 | 59.20098 | 0.5704053 | 44.61747 | 0.3435477 | 23.64511 |
| 205675_at | 4648246 | MTP | 0.8395886 | 91.71337 | 0.5778114 | 65.50542 | 0.3566993 | 40.59877 |
| 220507_s_at | 56550123 | UPB1 | 0.8866968 | 366.9551 | 0.5929381 | 275.2925 | 0.3567524 | 169.4148 |
| 230914_at | 3250320 | HNF4A | 0.9354432 | 386.6656 | 0.5893555 | 252.7635 | 0.364981 | 164.314 |
| 1556737_at | 16550598 | FLJ31222 | 0.9307335 | 229.5063 | 0.5741502 | 149.0377 | 0.3659484 | 98.07027 |
| 221008_s_at | 37574041 | AGXT2L1 | 1.0320444 | 111.4727 | 0.6355591 | 67.68906 | 0.3702034 | 44.63439 |
| 206119_at | 4502406 | BHMT | 0.94769424 | 7676.682 | 0.6510792 | 5581.056 | 0.3724005 | 3648.368 |
| 220424_at | 7657614 | NPHS2 | 1.100856 | 1354.614 | 0.7461829 | 940.7261 | 0.3725893 | 556.6851 |
| 227560_at | 31068337 | SFXN2 | 1.0103384 | 893.3289 | 0.5784623 | 530.699 | 0.3744776 | 346.8495 |
| 206878_at | 21536469 | DAO | 0.9553176 | 626.5679 | 0.6417686 | 481.6281 | 0.3774832 | 285.3359 |
| 203914_x_at | 52851450 | HPGD | 1.0108308 | 985.5264 | 0.59918 | 580.9052 | 0.3790265 | 412.1836 |
| 206172_at | 26787976 | IL13RA2 | 1.0571084 | 71.9148 | 0.577031 | 37.93879 | 0.3791732 | 25.28721 |
| 226030_at | 10363759 | ACADSB | 0.9469687 | 1112.143 | 0.5831609 | 706.57 | 0.3798742 | 498.7044 |
| 207076_s_at | 1.13E+08 | ASS | 0.9819452 | 7807.964 | 0.6110398 | 5191.742 | 0.3831461 | 3492.054 |
| 207387_s_at | 42794761 | GK | 0.8964044 | 422.3916 | 0.5932698 | 289.0754 | 0.3862093 | 191.5984 |
| 205666_at | 4503754 | FMO1 | 0.9907695 | 3028.701 | 0.6607965 | 2146.079 | 0.3914905 | 1401.319 |
| 241703_at | 5664001 | RPIB9 | 0.88089246 | 97.8649 | 0.6218046 | 68.66184 | 0.3917154 | 45.38718 |
| 220951_s_at | 20357571 | ACF | 0.8949415 | 383.9646 | 0.6314479 | 278.6698 | 0.3933726 | 186.0245 |
| 202847_at | 66346720 | PCK2 | 0.93247294 | 1126.112 | 0.690823 | 917.0765 | 0.3948821 | 547.5838 |
| 213967_at | 4685862 | LOC138046 | 0.9889094 | 300.984 | 0.6452329 | 208.6667 | 0.400118 | 133.9979 |
| 230278_at | 6505915 | TM7SF1 | 0.89933217 | 84.29004 | 0.6531568 | 54.40468 | 0.4036557 | 35.67263 |
| 205307_s_at | 52851407 | KMO | 0.9960288 | 188.2769 | 0.7433312 | 156.1696 | 0.405907 | 88.25554 |
| 204289_at | 13293226 | C14orf45 | 1.0482601 | 78.70741 | 0.6245744 | 49.32914 | 0.4106754 | 33.71832 |
| 206030_at | 73622272 | ASPA | 0.91991323 | 1038.339 | 0.6737331 | 770.0153 | 0.4121923 | 496.3843 |
| 223699_at | 13279082 | CNDP1; | 0.96248543 | 155.6105 | 0.6801313 | 110.4251 | 0.430668 | 74.8282 |
| | | CN1; CPGL2; | | | | | | |
| | | HsT2308; | | | | | | |
| | | MGC10825 | | | | | | |
| 218796_at | 1.17E+08 | C20orf42 | 0.96682906 | 234.7949 | 0.6667058 | 172.3095 | 0.4333774 | 112.3206 |
| 219840_s_at | 6319191 | TCL6 | 1.1816825 | 66.77751 | 0.7171395 | 40.14151 | 0.4368897 | 21.70967 |
| 219655_at | 13376041 | C7orf10 | 0.9589627 | 409.8573 | 0.6922788 | 294.1424 | 0.4369183 | 201.9344 |
| 241401_at | 13458148 | FBI4 | 1.0567125 | 125.1322 | 0.8619134 | 99.80727 | 0.4762132 | 60.14835 |
| 228293_at | 5763726 | LOC91614 | 1.1987456 | 260.9677 | 0.8210194 | 163.4289 | 0.4942515 | 109.3222 |
| 223599_at | 12407390 | TRIM6 | 1.0254507 | 614.8964 | 1.5350237 | 850.6952 | 0.5854892 | 356.9431 |

### Example 2

### Identifying NK Cell Associated Transcripts that are Increased in Renal Antibody-Mediated Rejection Compared to T Cell-Mediated Rejection

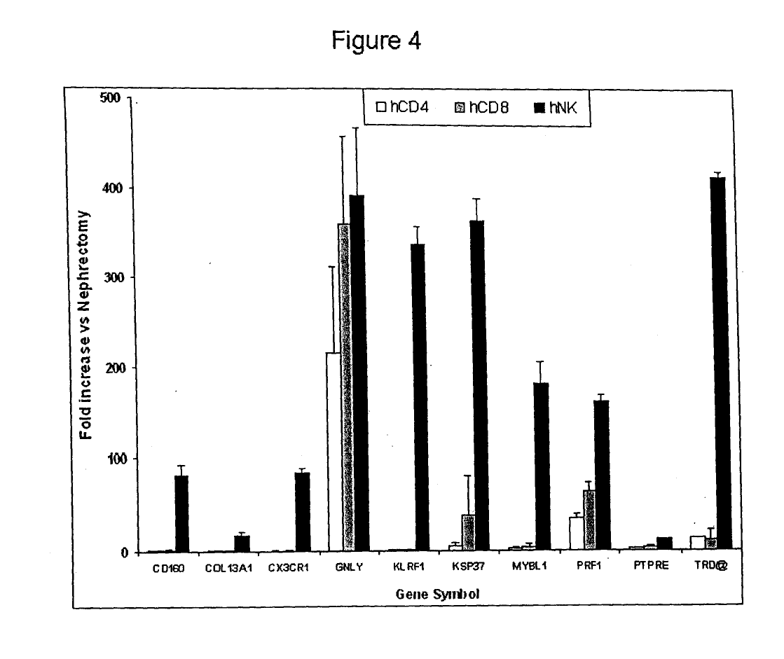

It was hypothesized that ABMR would be characterized by the presence of Fc receptor-positive NK cells, which mediate antibody-dependent cellular cytotoxicity. Using Affymetrix microarrays, a set of NK associated transcripts (NKATs) was generated (see Example 1). The NKATs were expressed at higher levels in purified human NK cells than in B cells, THP-1 monocytes, and nephrectomy biopsy samples. Expression levels of NKATs were compared in renal allograft biopsies from 26 cases of TCMR and 12 cases of ABMR with diffuse C4d staining. TCMR and ABMR cases were normalized to 10 living donor (LD) biopsies, and mixed ABMR/TCMR cases were excluded. Ten NKATs had significantly higher expression levels in ABMR biopsies compared to TCMR biopsies (p<0.05; Table 9). The ten NKATs (listed in Table 9) also had high expression levels in NK cells and low expression levels in LD biopsies. Expression of the ten NKATs was confirmed to be higher in NK cells than in CD4 or CD8 effector T cells (FIG. 4). With the exception of CX3CR1, the remaining NKATs were not expressed in neutrophils.

These data suggest that NK cells are recruited to a greater extent in ABMR than in TCMR. The increased expression of the NK transcripts suggests a role for NK cells in the pathogenesis of ABMR, and can be useful in differentiating ABMR from TCMR in renal allografts.

TABLE 9
NKATs and their expression levels in ABMR and TCMR biopsies

| Gene | | | | | | |
|---|---|---|---|---|---|---|
| Symbol | GI Number | LD | ABMR* | StdErr | TCMR* | StdErr |
| CD160 | 51702223 | 1 | 1.83 | 0.09 | 1.32 | 0.07 |
| COL13A1 | 22027564 | 1 | 2.1 | 0.21 | 1.28 | 0.05 |
| CX3CR1 | 3254346 | 0.95 | 12.45 | 1.8 | 5.45 | 0.76 |
| GNLY | 189229 | 1 | 4.5 | 0.54 | 2.09 | 0.29 |
| KLRF1 | 7705573 | 0.98 | 3.06 | 0.25 | 1.53 | 0.12 |
| KSP37 | 13442781 | 1 | 2.45 | 0.33 | 1.24 | 0.08 |
| MYBL1 | 10036935 | 1 | 2.06 | 0.18 | 1.37 | 0.1 |
| PRF1 | 133908619 | 1 | 5.26 | 0.66 | 2.88 | 0.38 |
| PTPRE | 30268344 | 0.96 | 4.84 | 0.41 | 2.73 | 0.39 |
| TRDa | 37003 | 1 | 5.95 | 0.56 | 3.1 | 0.42 |

*Fold increase over LD biopsies.

### Example 3

### Antibody-Mediated Rejection of Kidney Transplants Defined by Endothelial Changes

119 endothelial cell-associated transcripts (ENDATs) were identified from the literature. 173 consecutive renal allograft biopsies for cause, taken one week to 31 years post-transplant, were studied using microarrays to examine the relationship of ENDAT expression to circulating HLA-antibody, pathology, and outcome.

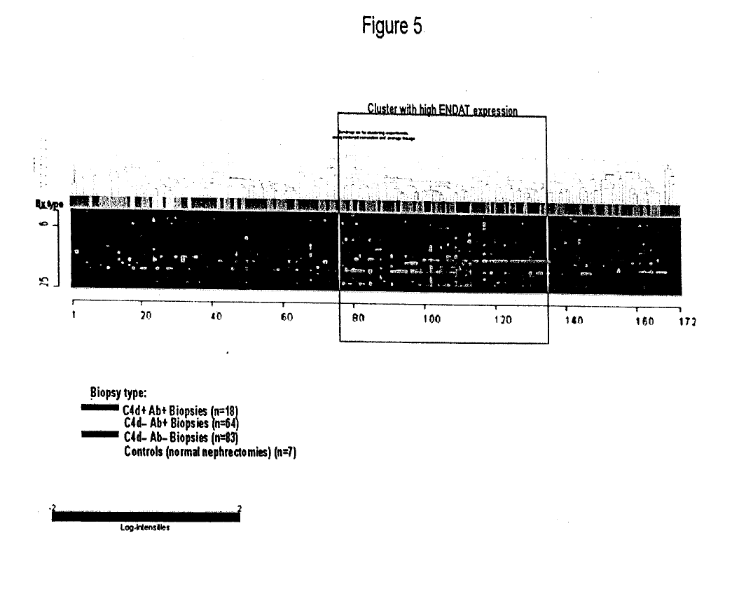

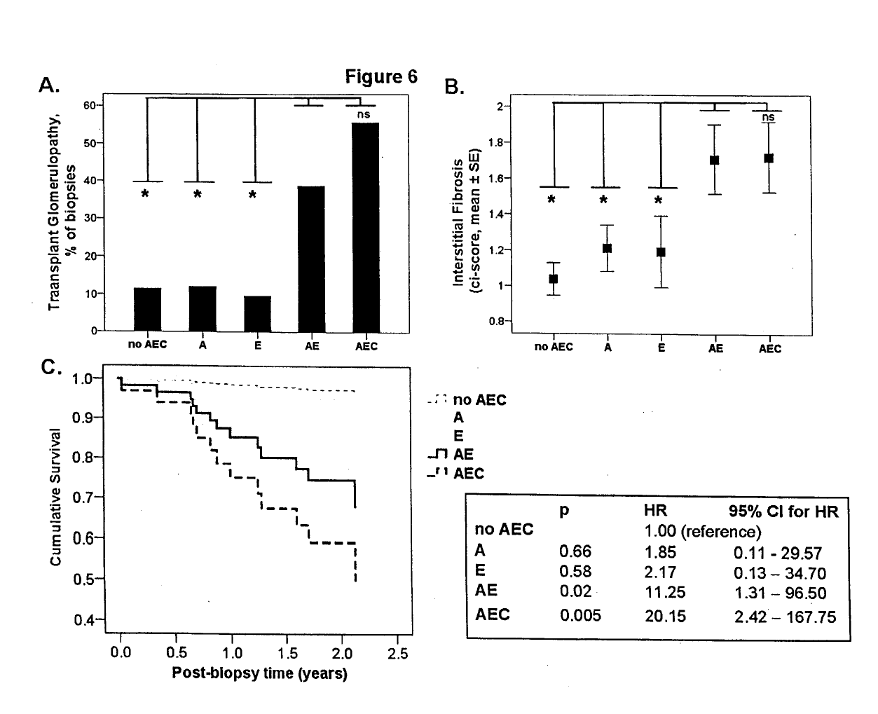

Mean ENDAT expression was increased in C4d+ ABMR and correlated with pathologic lesions of ABMR. 17 individual ENDATs were increased in C4d+ ABMR vs. T cell-mediated rejection, and many were associated with increased graft failure. The most increased ENDAT was von Willebrand's factor (VWF). Hierarchical clustering of all cases by ENDAT expression identified 31 C4d negative Ab+ biopsies that resembled C4d+ ABMR biopsies (FIG. 5). These C4d negative Ab+ biopsies with increased VWF had a phenotype similar to C4d+ ABMR, including transplant glomerulopathy, increased interstitial fibrosis, and increased graft loss (FIG. 6).

In kidney transplant biopsies for cause, increased expression of ENDATs such as VWF identifies many cases of ABMR that are C4d negative and missed by current criteria. Most kidney graft losses in this study were due to either C4d positive ABMR or to the newly recognized phenotype of C4d negative ABMR.

The following table (Table 10) lists individual endothelial genes that are differentially expressed between C4d+ antibody-mediated rejection and C4d− T cell-mediated rejection.

TABLE 10

| | | Controls | | C4d+ ABMR | | C4d− TCMR | |
|---|---|---|---|---|---|---|---|
| Gene Symbol | Gene Description | Normalized | Signal | Normalized | Signal | Normalized | Signal |

1) Endothelial genes increased in both ABMR and TCMR but more increased in ABMR:

| | | | | | | | |
|---|---|---|---|---|---|---|---|
| VWF | von Willebrand | 1.05 | 115.36 | 6.39 | 738.30 | 3.39 | 419.08 |
| | factor | | | | | | |
| CAV1 | caveolin 1, | 1.17 | 206.07 | 5.35 | 857.90 | 2.99 | 494.11 |
| | caveolae protein, | | | | | | |
| | 22 kDa | | | | | | |
| RHOJ | ras homolog gene | 0.99 | 27.60 | 2.94 | 85.27 | 1.65 | 53.58 |
| | family, member J | | | | | | |
| MCAM | melanoma cell | 1.12 | 192.50 | 2.93 | 476.93 | 2.02 | 326.45 |
| | adhesion molecule | | | | | | |
| CDH5 | cadherin 5, type 2, | 1.00 | 75.34 | 2.58 | 201.54 | 1.61 | 123.20 |
| | VE-cadherin | | | | | | |
| | (vascular | | | | | | |
| | epithelium) | | | | | | |
| SELE | selectin E | 1.06 | 23.25 | 2.23 | 55.26 | 1.20 | 28.66 |
| | (endothelial | | | | | | |
| | adhesion molecule | | | | | | |
| | 1) | | | | | | |
| PALMD | palmdelphin | 0.93 | 78.15 | 2.12 | 182.35 | 1.36 | 122.61 |
| PECAM1 | platelet/endothelial | 0.93 | 294.26 | 2.00 | 638.35 | 1.60 | 514.29 |
| | cell adhesion | | | | | | |
| | molecule (CD31 | | | | | | |
| | antigen) | | | | | | |
| CYYR1 | cysteine and | 1.04 | 162.34 | 1.52 | 238.68 | 1.15 | 182.55 |
| | tyrosine-rich 1 | | | | | | |
| CD34 | CD34 antigen | 1.05 | 138.33 | 1.50 | 198.87 | 1.12 | 148.89 |

2) Endothelial genes increased in ABMR but not changed in TCMR:

| | | | | | | | |
|---|---|---|---|---|---|---|---|
| KLF4 | Kruppel-like factor | 1.11 | 236.31 | 1.52 | 318.70 | 1.00 | 216.59 |
| | 4 (gut) | | | | | | |
| TEK | TEK tyrosine | 1.04 | 190.45 | 1.47 | 275.89 | 1.04 | 198.66 |
| | kinase, endothelial | | | | | | |
| | (venous | | | | | | |
| | malformations, | | | | | | |
| | multiple cutaneous | | | | | | |
| | and mucosal) | | | | | | |
| SOX18 | SRY (sex | 1.02 | 13.48 | 1.46 | 19.92 | 1.08 | 14.41 |
| | determining region | | | | | | |
| | Y)-box 18 | | | | | | |
| ZNF521 | zinc finger protein | 0.92 | 37.82 | 1.38 | 55.56 | 0.80 | 33.75 |
| | 521 | | | | | | |
| RASIP1 | Ras interacting | 0.98 | 80.80 | 1.37 | 114.53 | 0.97 | 80.99 |
| | protein 1 | | | | | | |
| HOXD4 | homeo box D4 | 1.02 | 372.75 | 1.33 | 495.27 | 1.02 | 378.73 |
| RAI14 | retinoic acid | 0.95 | 273.72 | 1.21 | 347.63 | 0.87 | 256.99 |
| | induced 14 | | | | | | |

3) Endothelial genes decreased in both ABMR and TCMR but more decreased in TCMR:

| | | | | | | | |
|---|---|---|---|---|---|---|---|
| EMCN | endomucin | 1.01 | 700.89 | 0.85 | 600.32 | 0.64 | 459.15 |
| KDR | kinase insert | 0.94 | 307.06 | 0.77 | 244.78 | 0.60 | 197.55 |
| | domain receptor | | | | | | |
| | (a type III receptor | | | | | | |
| | tyrosine kinase) | | | | | | |
| MAOB | monoamine | 0.97 | 1450.68 | 0.69 | 1031.30 | 0.52 | 795.14 |
| | oxidase B | | | | | | |

4) Endothelial genes decreased in ABMR but not changed in TCMR:

| | | | | | | | |
|---|---|---|---|---|---|---|---|
| FOXF2 | forkhead box F2 | 1.02 | 12.59 | 0.86 | 10.61 | 0.99 | 12.25 |
| CETP | cholesteryl ester | 1.04 | 26.61 | 0.72 | 18.27 | 1.06 | 29.67 |
| | transfer protein, | | | | | | |
| | plasma | | | | | | |

5) Endothelial genes not changed in ABMR but decreased in TCMR:

| | | | | | | | |
|---|---|---|---|---|---|---|---|
| PODXL | podocalyxin-like | 1.11 | 1489.49 | 1.06 | 1404.16 | 0.74 | 1067.37 |
| DLC1 | deleted in liver | 0.95 | 273.83 | 0.98 | 287.39 | 0.74 | 221.40 |
| | cancer 1 | | | | | | |
| FGD5 | FYVE, RhoGEF | 0.88 | 212.06 | 0.97 | 233.58 | 0.71 | 170.90 |
| | and PH domain | | | | | | |
| | containing 5 | | | | | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims

What is claimed is:

1. A method for detecting tissue rejection, said method comprising determining whether or not tissue transplanted into a mammal contains cells having a transplant rejection profile, wherein the presence of said cells indicates that said tissue is being rejected.

2. The method of claim 1, wherein said mammal is a human.

3. The method of claim 1, wherein said tissue is kidney tissue.

4. The method of claim 1, wherein said tissue is a kidney.

5. The method of claim 1, wherein said method comprises using kidney cells obtained from a biopsy to assess the presence or absence of said transplant rejection profile.

6. The method of claim 1, wherein said determining step comprises analyzing nucleic acids.

7. The method of claim 1, wherein said determining step comprises analyzing polypeptides.

8. A method for distinguishing antibody-mediated rejection and T cell-mediated rejection, said method comprising determining whether or not tissue transplanted into a mammal contains cells having an ABMR expression profile, wherein the presence of said cells indicates that said tissue is undergoing antibody-mediated rejection.

9. The method of claim 8, wherein said mammal is a human.

10. The method of claim 8, wherein said tissue is kidney tissue.

11. The method of claim 8, wherein said tissue is a kidney.

12. The method of claim 8, wherein said method comprises using kidney cells obtained from a biopsy to assess the presence or absence of said ABMR expression profile.

13. The method of claim 8, wherein said determining step comprises analyzing nucleic acids.

14. The method of claim 8, wherein said determining step comprises analyzing polypeptides.

15. A method for distinguishing antibody-mediated rejection and T cell-mediated rejection, said method comprising determining whether or not tissue transplanted into a mammal contains cells having a TCMR expression profile, wherein the presence of said cells indicates that said tissue is undergoing T cell-mediated rejection.

16. The method of claim 15, wherein said mammal is a human.

17. The method of claim 15, wherein said tissue is kidney tissue.

18. The method of claim 15, wherein said tissue is a kidney.

19. The method of claim 15, wherein said method comprises using kidney cells obtained from a biopsy to assess the presence or absence of said TCMR expression profile.

20. The method of claim 15, wherein said determining step comprises analyzing nucleic acids.

21. The method of claim 15, wherein said determining step comprises analyzing polypeptides.
